(19) United States
(12) Patent Application Publication
(10) Pub. No.: US 2022/0176168 A1
(43) Pub. Date: Jun. 9, 2022

(54) SMART MASKS

(71) Applicant: XUPERMASK OPCO, LLC, Los Angeles, CA (US)

(72) Inventor: William Adams, Los Angeles, CA (US)

(21) Appl. No.: 17/300,298

(22) Filed: Dec. 3, 2020

(51) Int. Cl. A62B 7/10; A62B 18/00; A62B 18/08; A62B 23/02; A62B 9/00; A61L 2/26; A61L 2/10; B01D 46/24; B01D 46/00; B01D 46/44; B01D 46/42

(52) U.S. Cl. CPC A62B 7/10; H04R 1/028; A62B 18/084; A62B 23/02; A62B 18/08; A62B 9/00; A61L 2/26; A61L 2/10; B01D 46/2411; B01D 46/002; B01D 46/44; B01D 46/4245; B01D 46/0043; A61L 2202/11; A61L 2202/26; A61L 2202/14; A62B 18/006

(57) ABSTRACT

A smart mask includes a face covering, an attachment member, one or more power source(s), an air circulation subsystem, an exhaust subsystem, and a controller. The face covering is configured to cover a face area of a wearer. The attachment member is configured to attach the face covering onto the face area of the wearer. The air circulation subsystem is powered by at least one of the one or more power source(s) and configured to filter outside air and draw the filtered outside air into the face area. The exhaust subsystem is placed below the air circulation subsystem and configured to purge exhaled air out of the face area. The controller is configured to control the air circulation subsystem.

### BACKGROUND

Protective respirators or masks are pieces of fabric, kits, or equipment worn on the head and face to protect the wearer from inhaling hazardous atmospheres, including fumes, vapors, gases, or particulate matter such as dust and airborne microorganisms. Protective respirators or masks come in many different configurations and ratings. The primary ratings are N, P, and R. Following the letter rating is a number, generally, 95, 99, or 100, which relates to how much the filter has been measured to remove of particulate matter of 0.3 microns in diameter or greater. “N” represents “Not oil resistant”, “R” represents “Resistant to oil”, and “P” represents “Oil Proof”. “95” indicates that the mask removes 95% of all particles that are at least 0.3 microns in diameter, “99” indicates that the mask removes 99% of all particles that are at least 0.3 microns in diameter, and “100” represents that the mask removes 99.97% of all particles that are at least 0.3 microns in diameter.

In the past, such protective masks are mostly worn by professionals (e.g., healthcare professionals and construction professionals). However, since the COVID-19 was declared a pandemic, many countries required their citizens to wear masks while in public during the pandemic. A growing number of U.S. states have also mandated the use of masks and face coverings while in public during the pandemic. Fabric masks and disposable paper masks are the most popular. However, many of these masks either do not have sufficient breathability or cannot provide sufficient protection. Wearing such a mask can also interfere with a user's capability of listening to music, answering phone calls, etc.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter.

The embodiments described herein are related to smart devices and protective respirators or masks (hereinafter referred to as smart masks) that are configured to protect a wearer from inhaling hazardous atmospheres.

The smart mask includes a face covering configured to cover a face area of a wearer and an attachment member configured to attach the face covering onto the face area of the wearer. The smart mask also includes one or more power source(s), an air circulation subsystem, and an exhaust subsystem. The air circulation subsystem is powered by at least one of the one or more power source(s) and configured to filter outside air and draw the filtered outside air into the face area. The exhaust subsystem is placed below the air circulation subsystem and configured to purge exhaled air out of the face area. The smart mask also includes a controller configured to control the air circulation subsystem.

In some embodiments, the air circulation subsystem includes one or more filter receptacle(s), one or more fan(s), and one or more puck(s). Each of the one or more filter receptacle(s) is configured to receive a filter cartridge for filtering the outside air drawn into the face area. Each of the one or more fan(s) is powered by at least one of the one or more power source(s) and configured to draw outside air through the corresponding filter cartridge into the face area. Each of the puck(s) is configured to cover the corresponding filter cartridge or fan. In some embodiments, each fan is configured to operate at different speeds (e.g., low, medium, high, etc.). The controller is configured to control a speed of each fan.

In some embodiments, the one or more fan(s) include a left fan placed on a left side of the face covering and a right fan placed on a right side of the face covering. The one or more filter receptacle(s) includes a left filter receptacle and a right filter receptacle. The left receptacle is coupled to the left fan and configured to receive a left filter cartridge, and the right receptacle is coupled to the right fan and configured to receive a right filter cartridge. The one or more puck(s) includes a left puck and a right puck. The left puck is configured to cover the left fan and the left filter cartridge, and the right puck is configured to cover the right fan and the right filter cartridge.

In some embodiments, each filter receptacle includes a magnetic portion configured to hold a metal frame of the corresponding filter cartridge in place magnetically. In some embodiments, each filter receptacle has a circular-shaped recess configured to receive a circular-shaped filter cartridge.

In some embodiments, the smart mask further includes an audio subsystem. The audio subsystem is powered by at least one of the one or more power source(s) and configured to receive a sound signal wirelessly from a user terminal. The controller is further configured to control the audio subsystem. In some embodiments, the audio subsystem includes at least a pair of earbuds, or a pair of speakers. In some embodiments, each of the pair of earbuds includes a magnetic portion or a metal portion, and the attachment member also includes a pair of magnetic portion or metal portions corresponding to the pair of earbuds, such that when the pair of earbuds are not in use, each earbud is configured to be secured to the corresponding magnetic portion or metal portion of the attachment member magnetically.

In some embodiments, the audio subsystem also includes a microphone configured to receive a voice input. The controller is further configured to process the voice input to identify one or more voice command(s) for controlling the air circulation subsystem or the audio subsystem.

In some embodiments, the audio subsystem also includes an active noise cancellation subsystem configured to detect surrounding noise and cause the audio subsystem emit a sound wave with a same amplitude of the surrounding noise but with inverted phase to the surrounding noise to cancel out the surrounding noise.

In some embodiments, the smart mask further includes a network interface configured to connect to a user terminal or a computer network. When the user terminal is connected to the network interface, the network interface is configured to transmit status data to the user terminal or a cloud storage, and/or to grant the user terminal control of the air circulation subsystem.

In some embodiments, the smart mask further includes a temperature sensor configured to detect a body temperature of the wearer. In yet some other embodiments, the smart mask further includes a heart rate monitor configured to detect a heart rate of the wearer.

In some embodiments, the one or more power source(s) include one or more rechargeable batteries, and the smart mask further includes a charging port (e.g., a USBC port, a micro USB port, etc.) configured to charge the one or more rechargeable batteries.

In some embodiments, the smart mask further includes one or more light sources (e.g., LEDs). In some embodiments, some of the one or more light sources are coupled to one or more controllers of the air circulation subsystem, the audio subsystem, and/or the active noise cancellation subsystem, indicating status of the corresponding subsystem. In some embodiments, some of the one or more light sources are coupled to the one or more pucks to create a light effect. In some embodiments, each ultraviolet light source is configured to emit light at different intensities. The controller is further configured to control an intensity of each light source.

In some embodiments, some of the one or more light sources are ultraviolet light sources. Each ultraviolet light source is powered by at least one of the one or more power source(s) and configured to disinfect the face covering and/or an area around each filter cartridge. The controller is further configured to control the one or more ultraviolet light source(s). In some embodiments, each ultraviolet light source is coupled to a corresponding puck or a corresponding filter receptacle. In some embodiments, one or more ultraviolet light(s) is positioned around each filter cartridge. In some embodiments, each ultraviolet light source is configured to emit ultraviolet light at different intensities. The controller is further configured to control an intensity of each ultraviolet light source.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and details through the use of the accompanying drawings in which:

FIG. 1A illustrates a wearer wearing an example smart mask that incorporates the principles described herein;

FIG. 1B illustrates a perspective view of the example smart mask;

FIG. 1C illustrates various technical components of the example smart mask;

FIG. 1D illustrates a front view of the example smart mask being worn by a wearer;

FIG. 1E illustrates a front view of the example smart mask not being worn;

FIG. 1F illustrates a right-side view of the example smart mask being worn by a wearer;

FIG. 1G illustrates a left-side view of the example smart mask being worn by a wearer;

FIG. 1H illustrates a right-side view of the example smart mask not being worn;

FIG. 1I illustrates a left-side view of the example smart mask not being worn;

FIG. 1J illustrates an enlarged view of a left controller and a right controller of the example smart mask;

FIG. 2A illustrates a back side view of a back frame of a face covering body of the example smart mask;

FIG. 2B illustrates a back side view of an back frame of the face covering body of the example smart mask;

FIG. 2C illustrates a perspective view of the back frame of the face covering body of the example smart mask;

FIG. 2D illustrates an example embodiment of the back frame of the face covering body;

FIG. 2E illustrates an example embodiment of the back frame of the face covering body;

FIG. 2F illustrates an enlarged back side view of a right side of the back frame of the face covering body;

FIG. 2G illustrates an enlarged back side view of a left corner of the back frame of the face covering body having a microphone embedded thereto;

FIG. 2H illustrates a front side view of a front frame of the face covering body;

FIG. 21 illustrates a perspective view of the front frame of the face covering body;

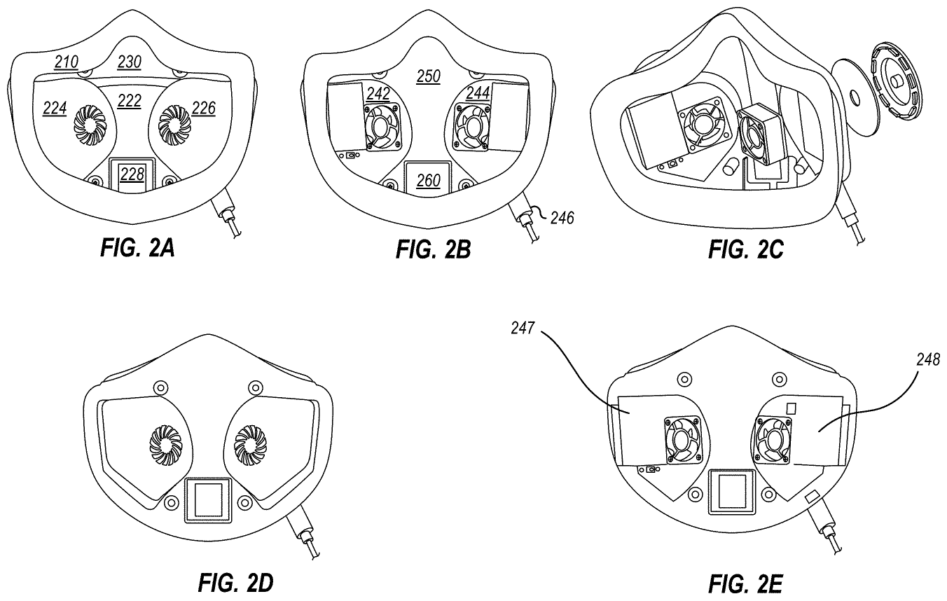

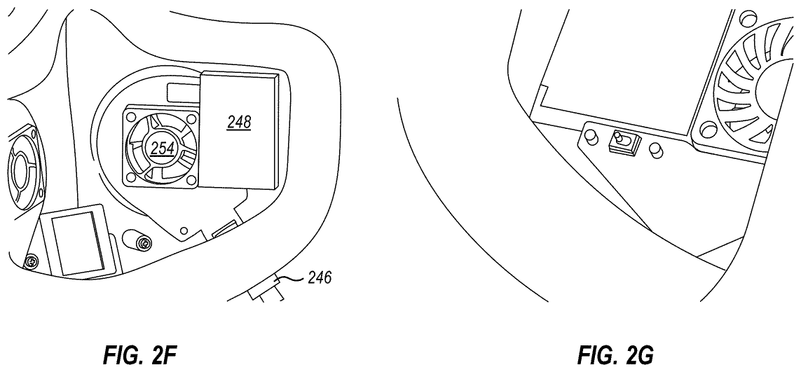

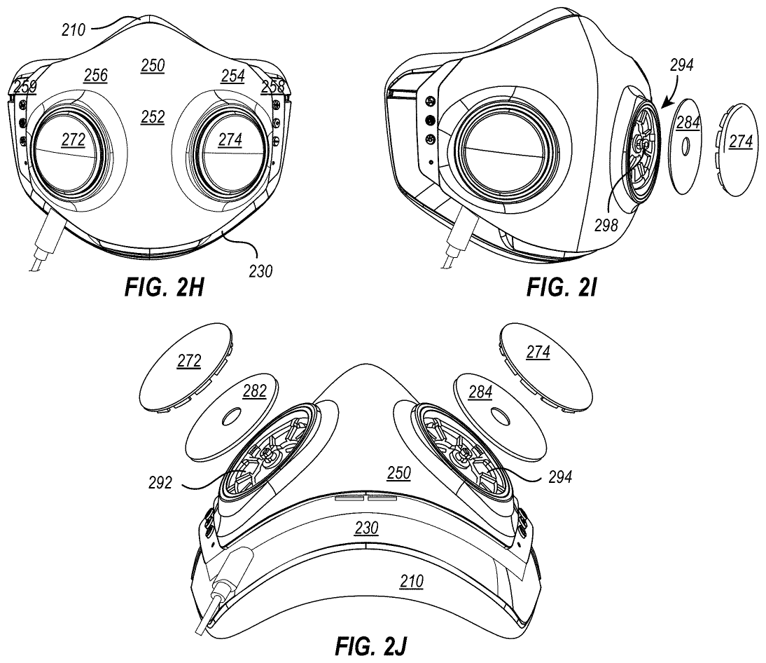

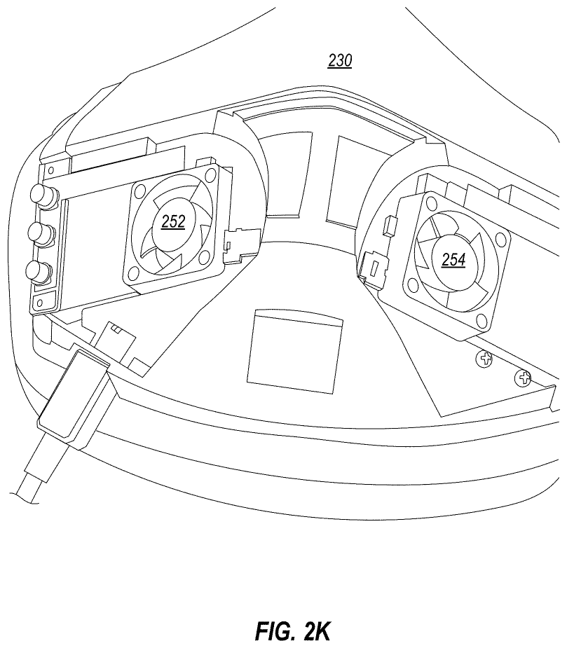

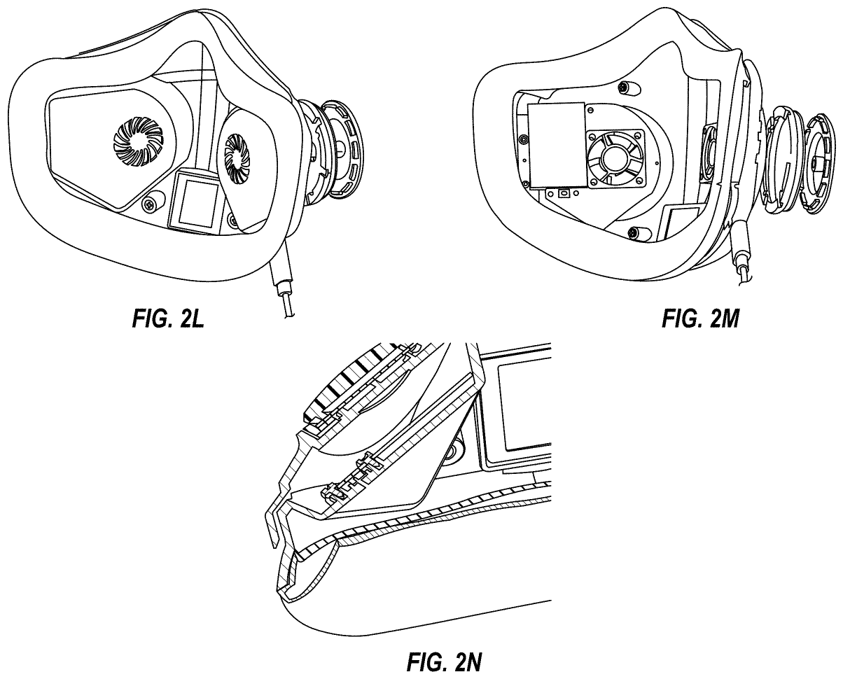

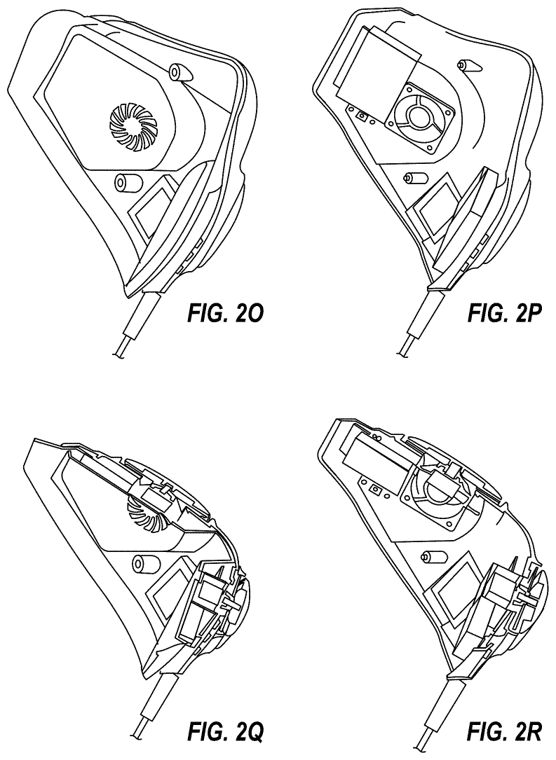

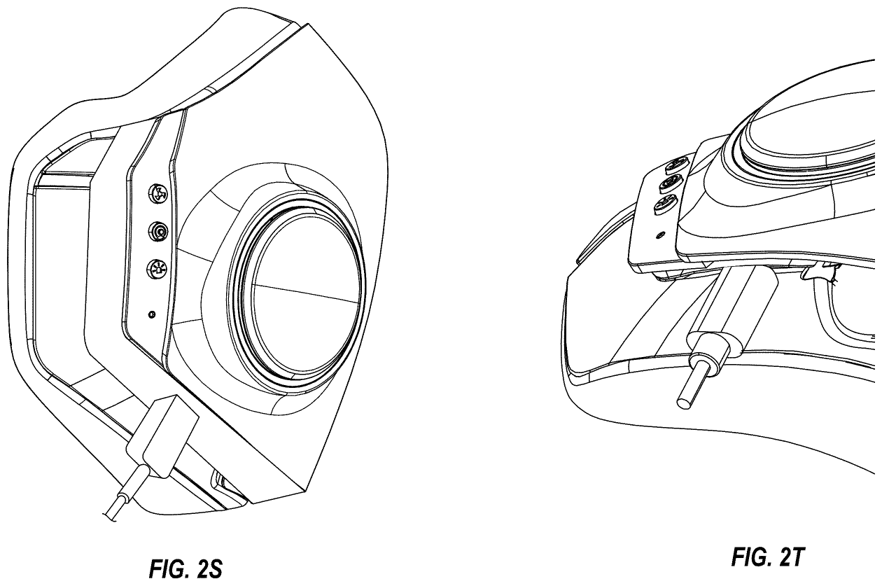

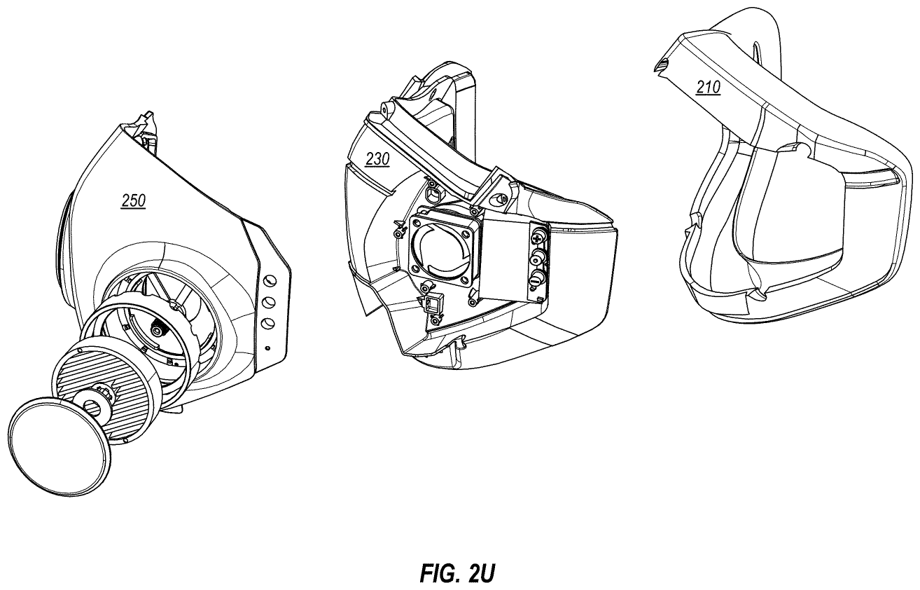

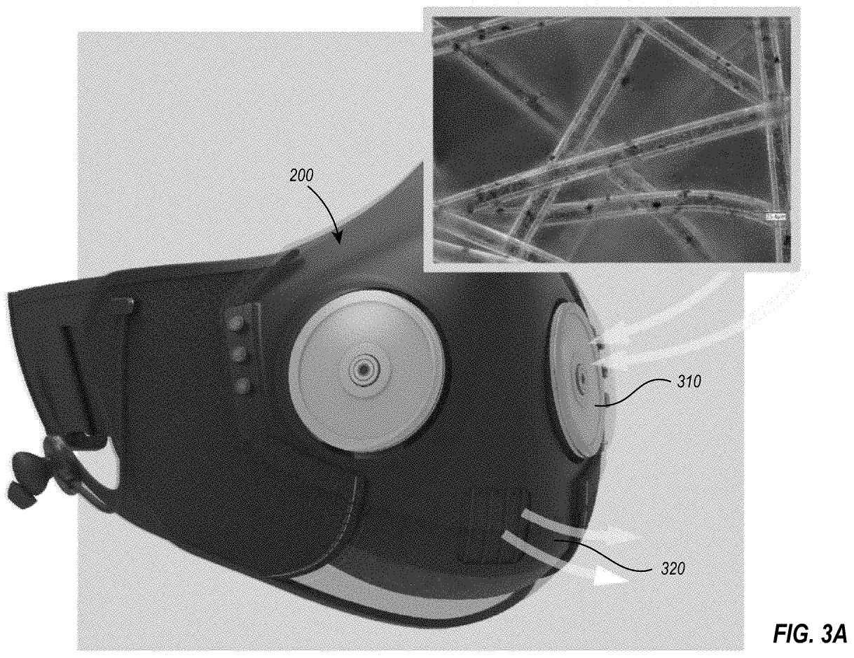

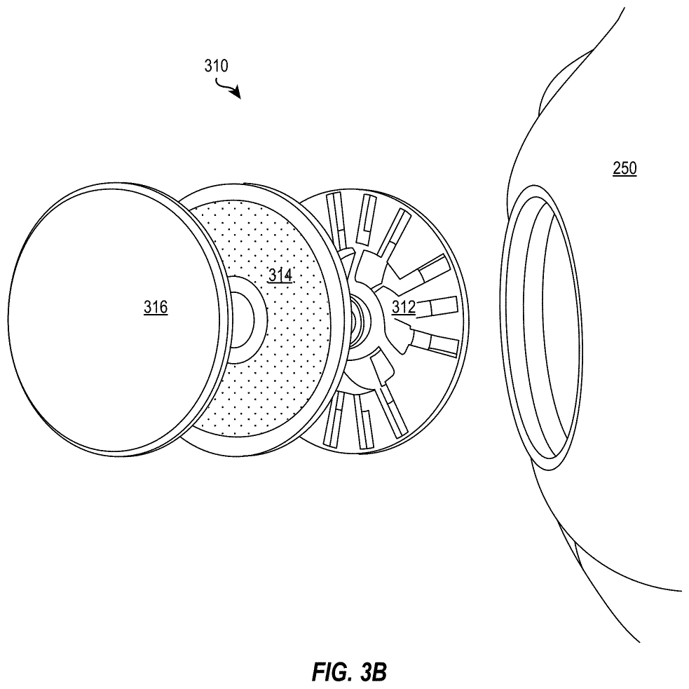

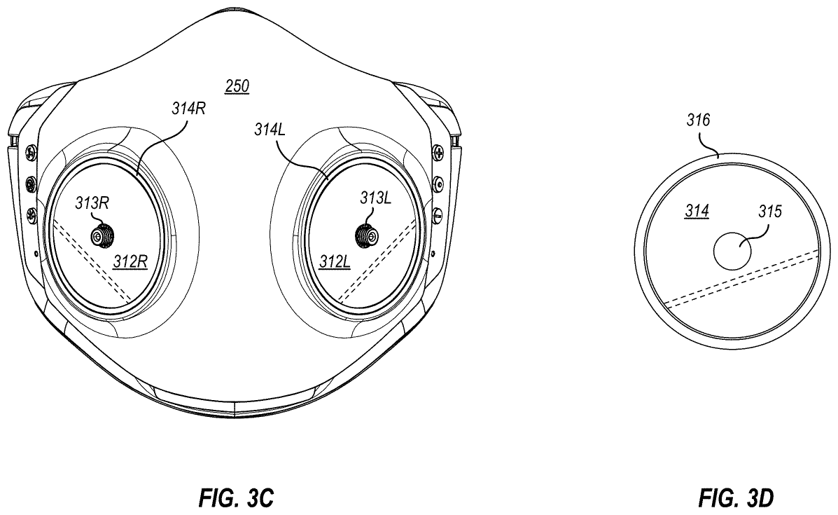

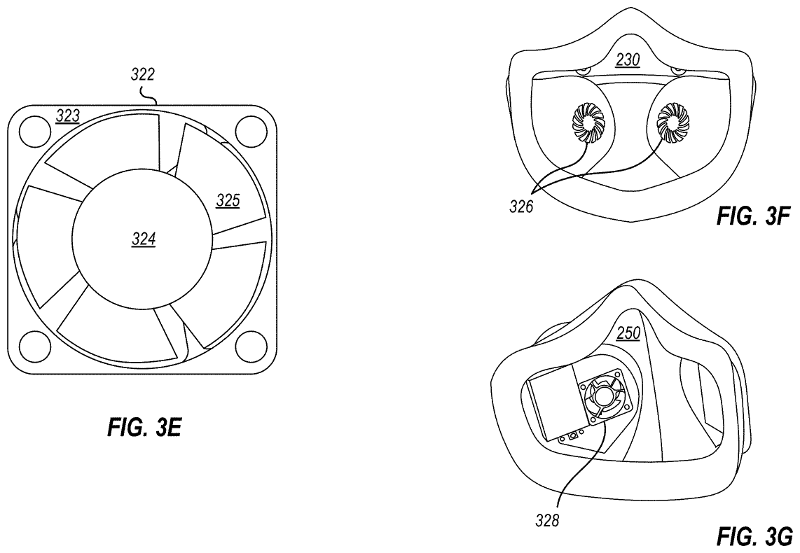

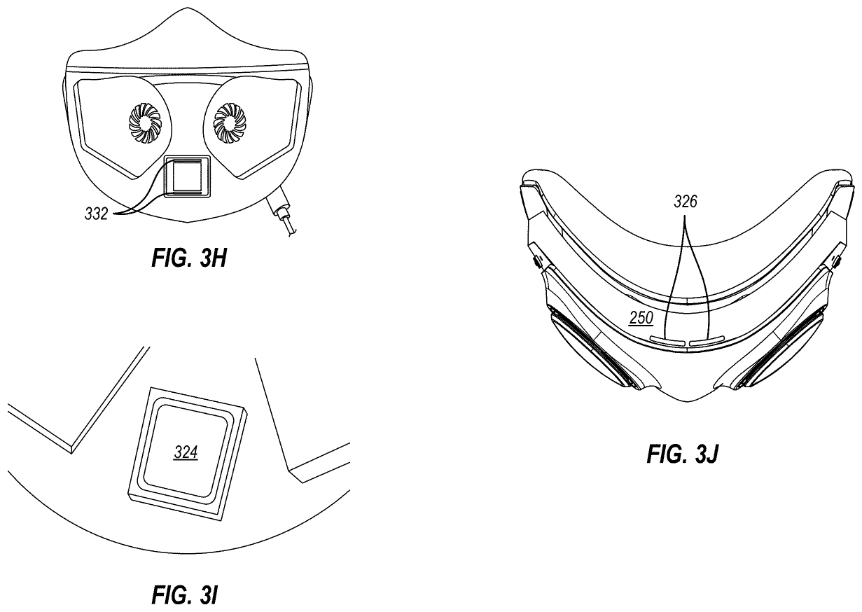

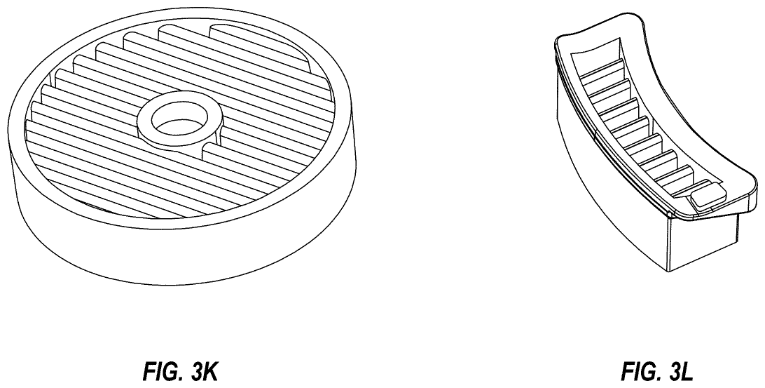

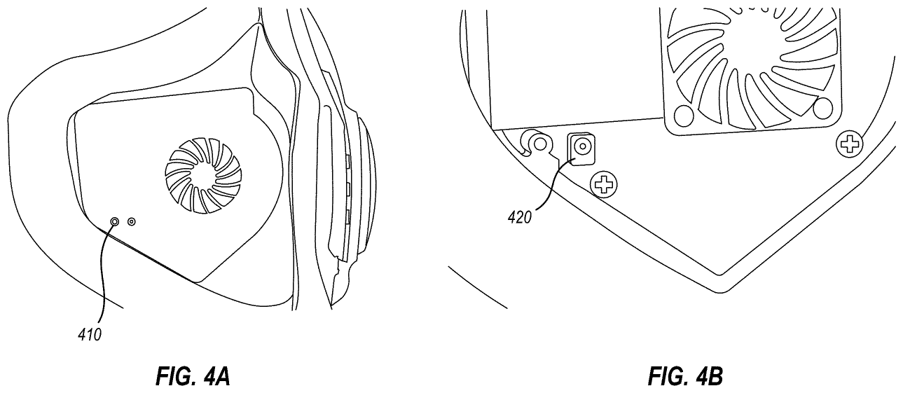

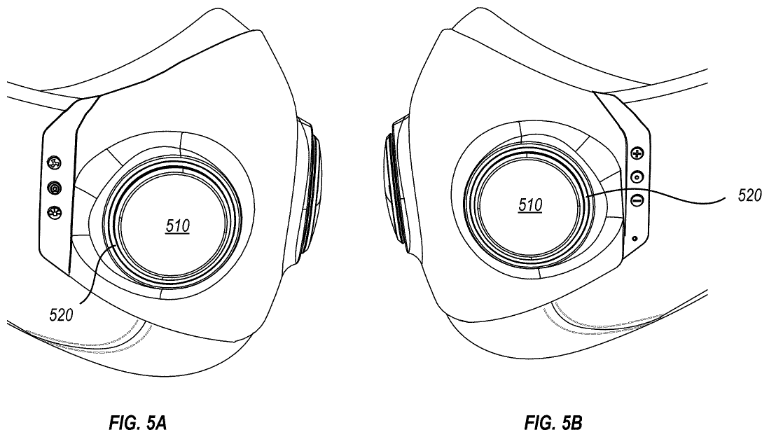

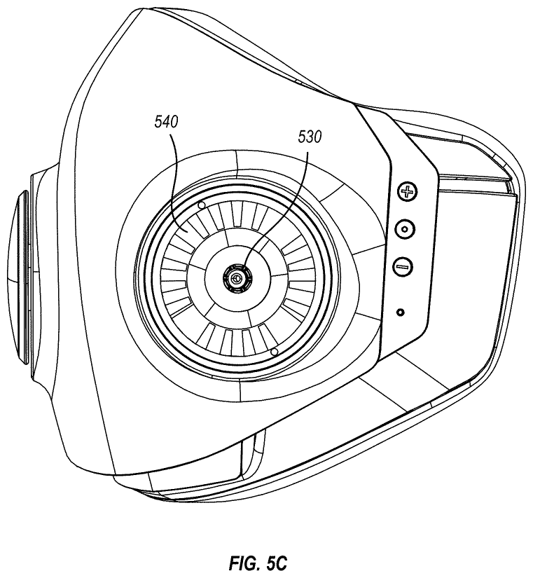

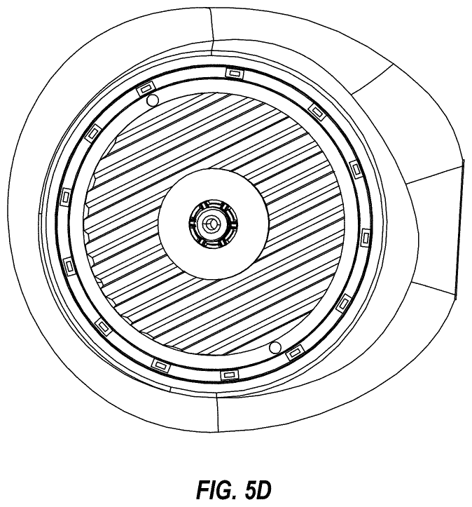

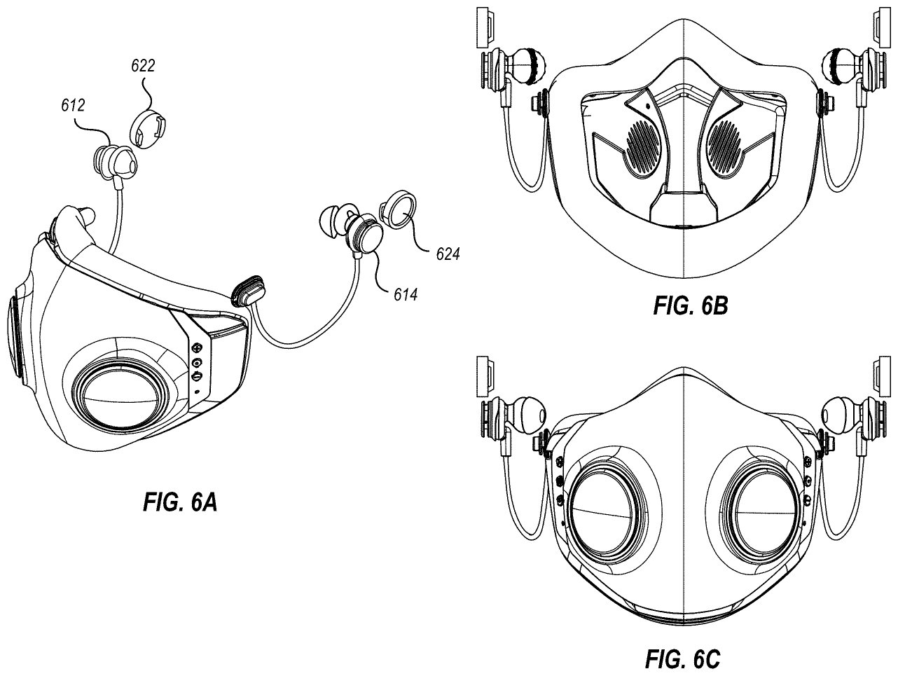

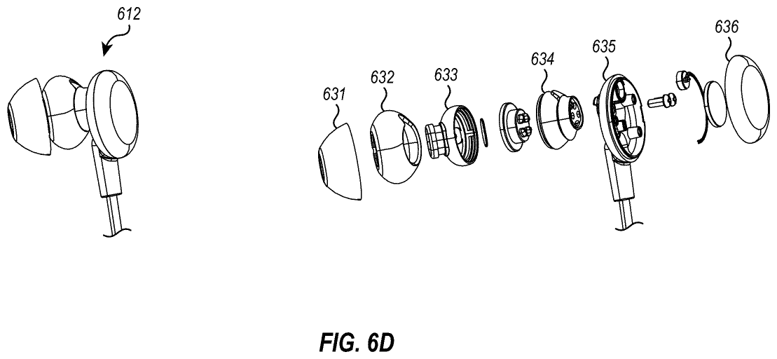

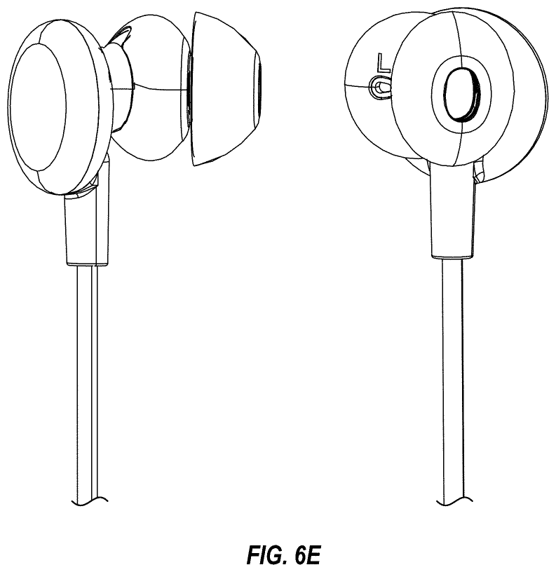

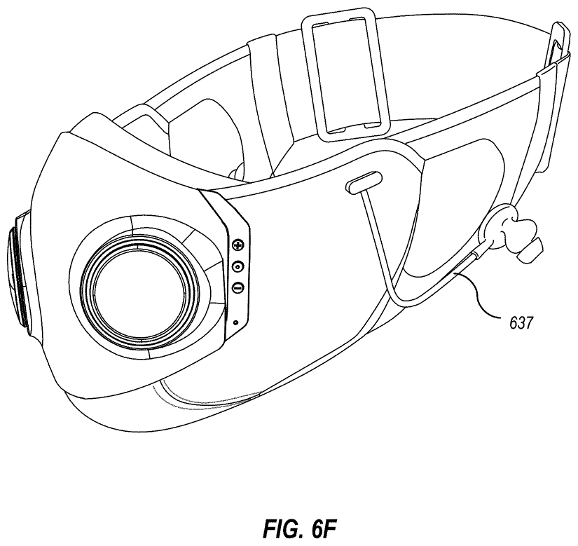

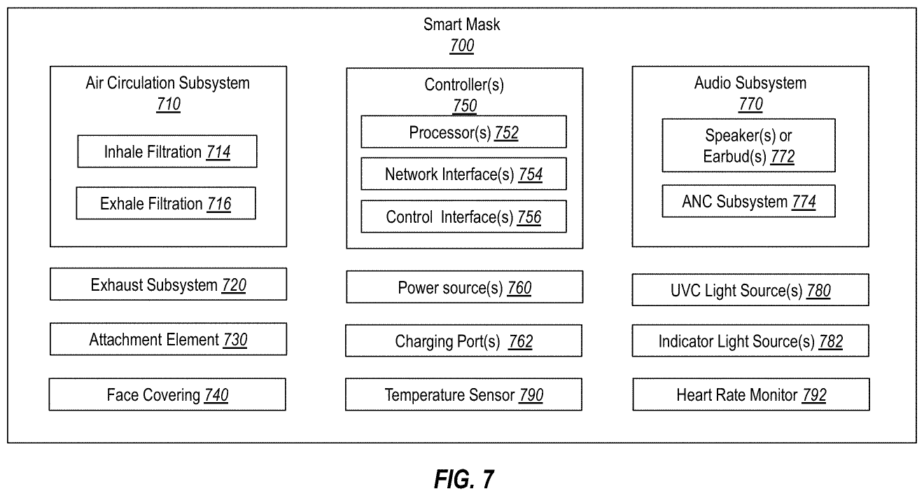

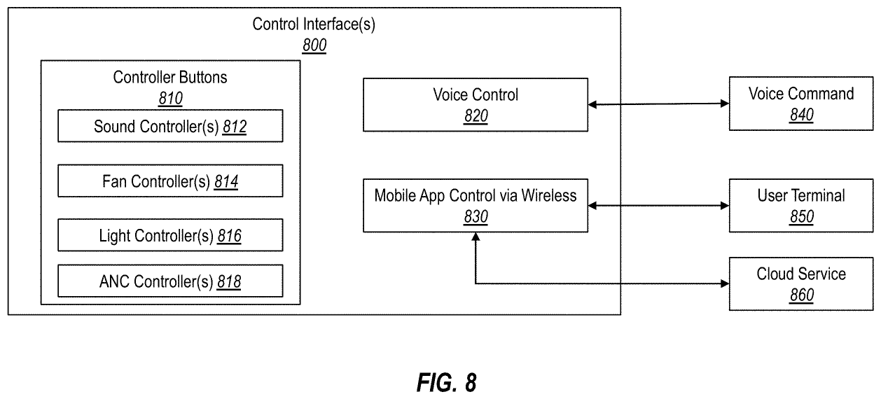

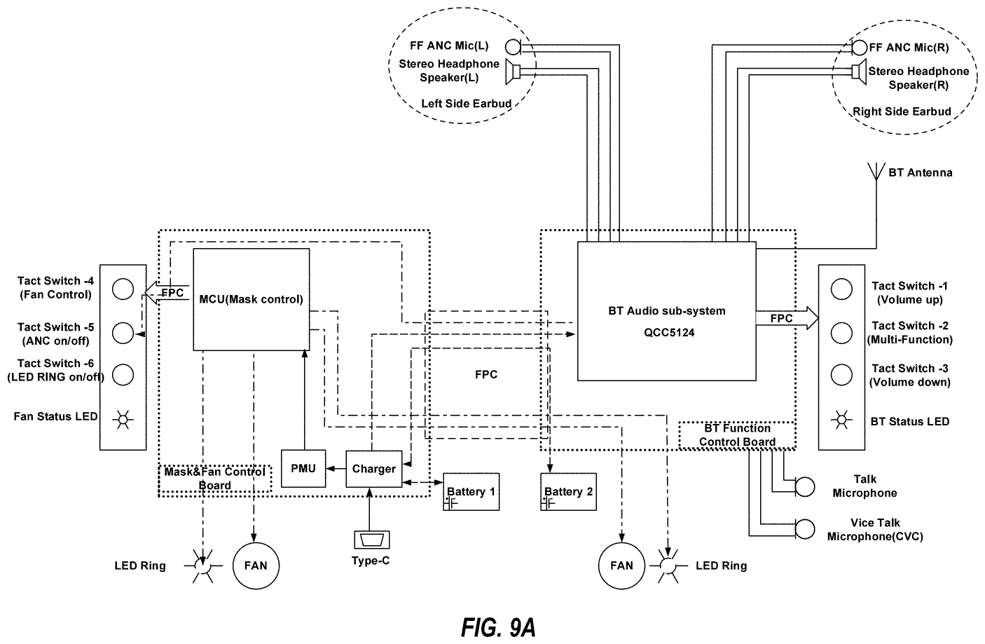

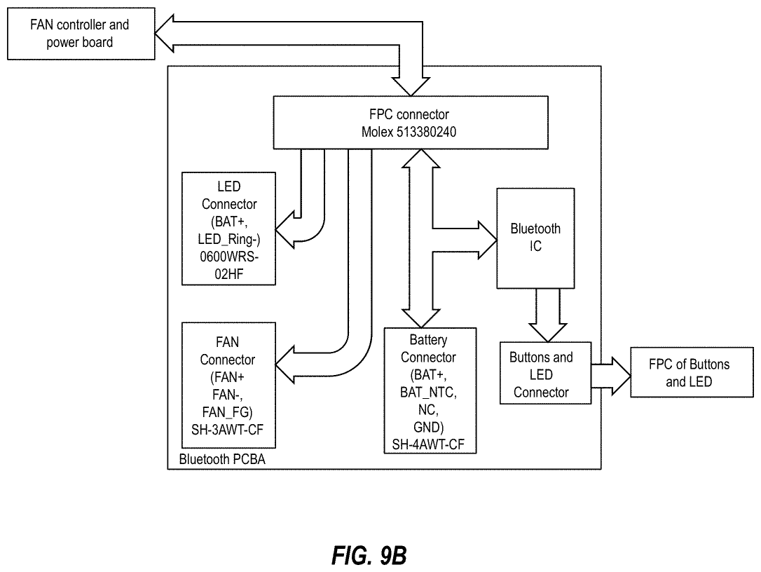

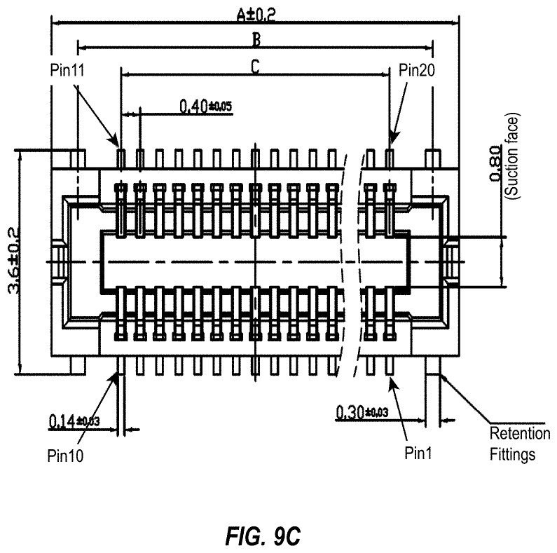

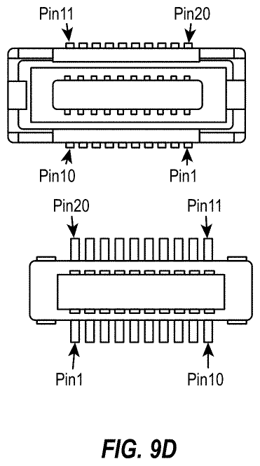

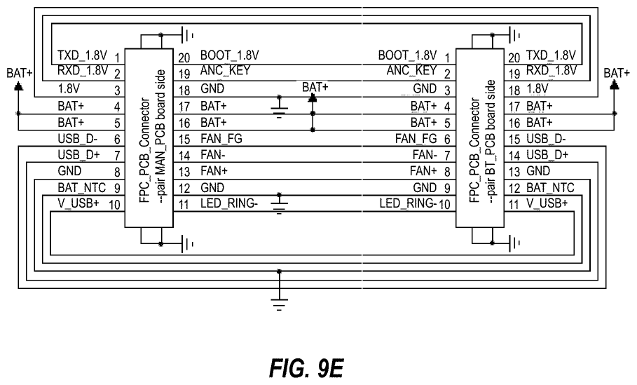

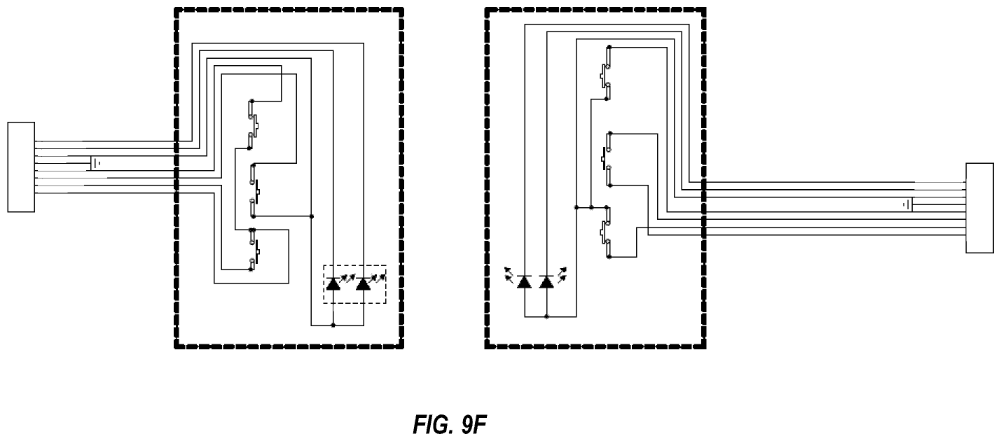

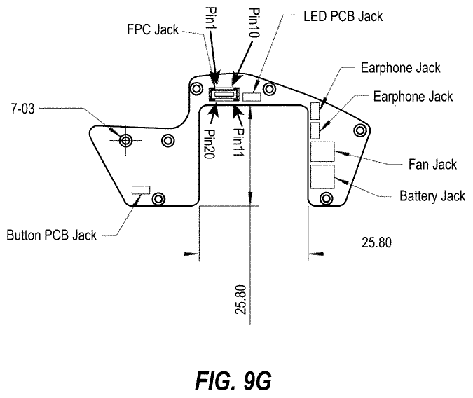

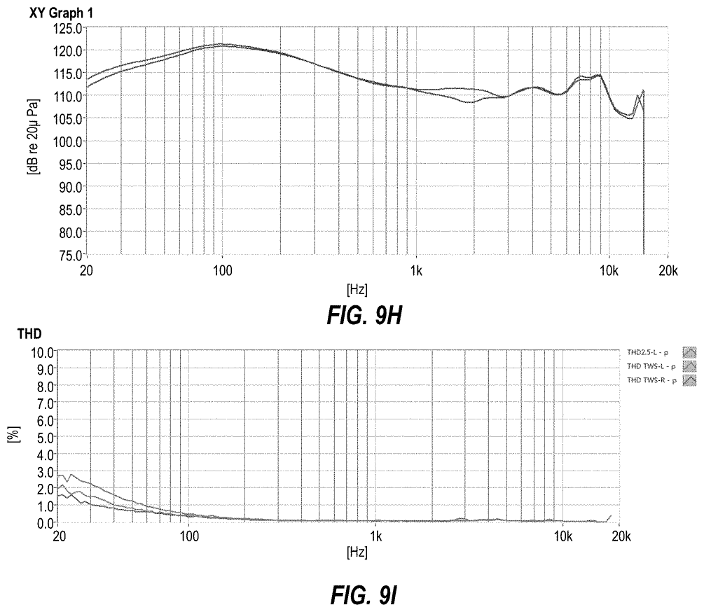

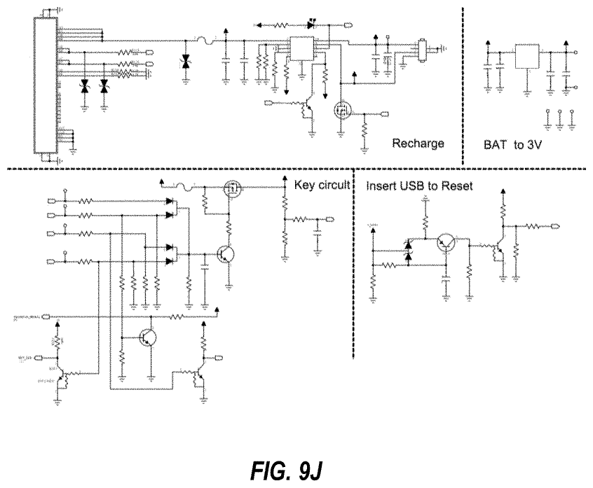

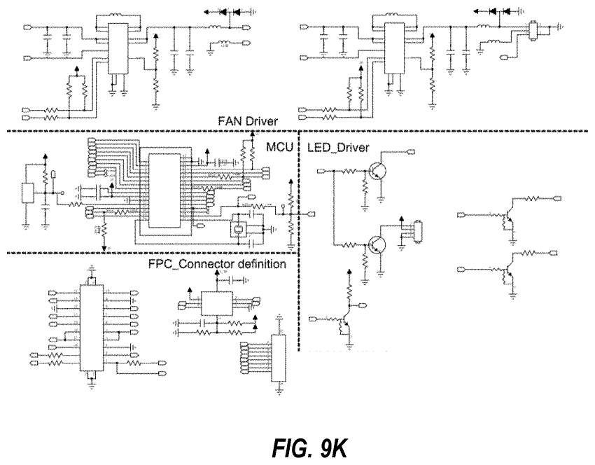

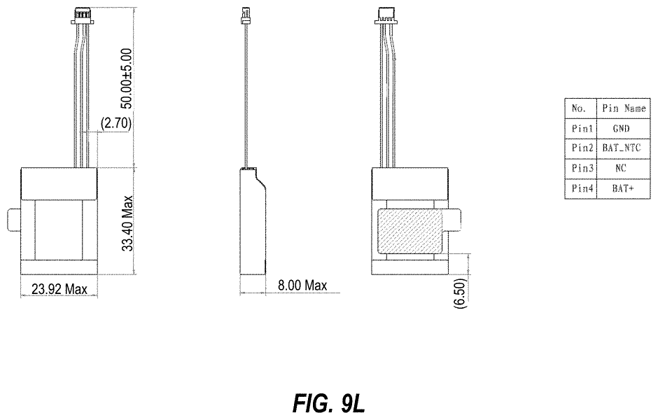

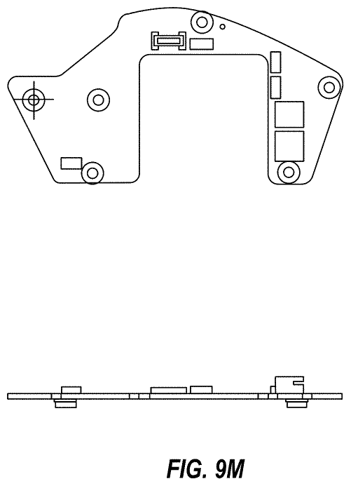

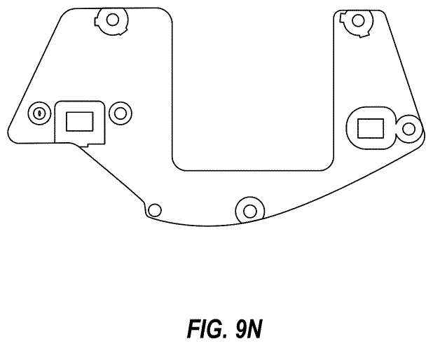

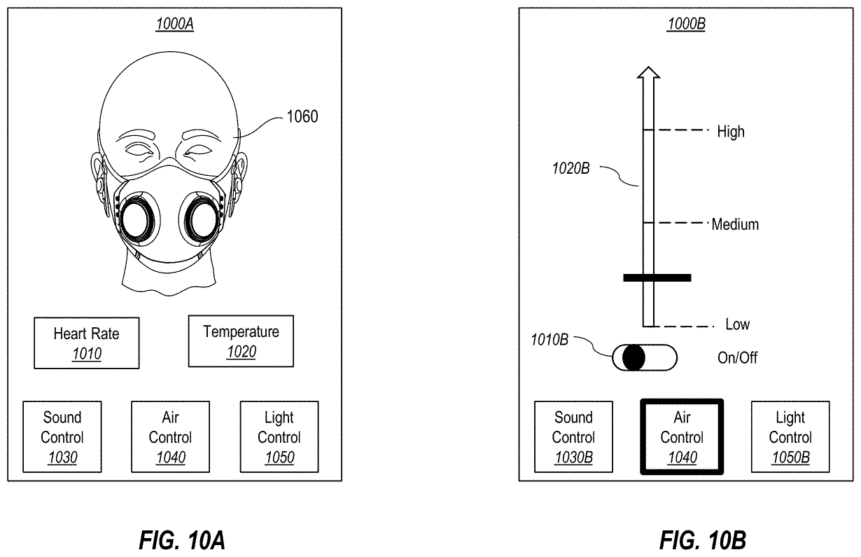

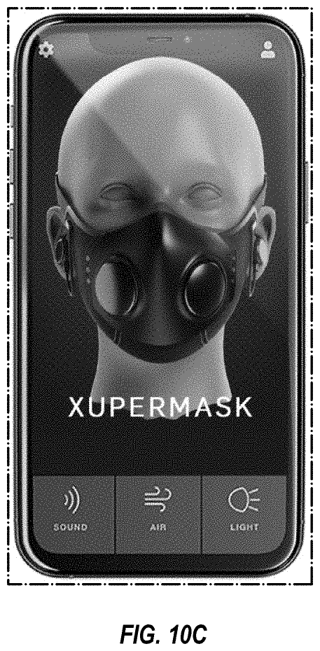

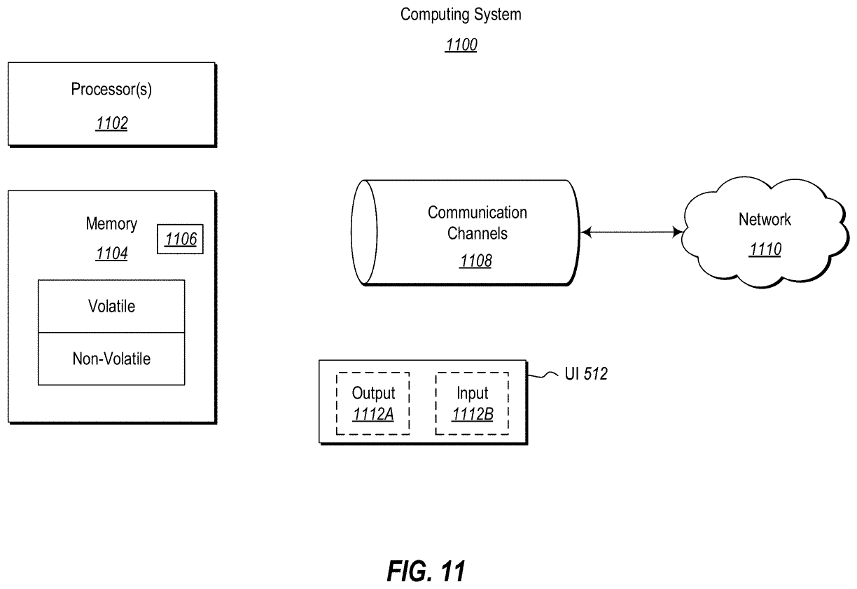

FIG. 2J illustrates a bottom side view of the front frame of the face covering body;

FIG. 2K illustrates a front side view of the back frame of the face covering body;

FIG. 2L illustrates a back side view of the face covering body including the back frame;

FIG. 2M illustrates a back side view of the face covering body with the back frame being removed;

FIG. 2N illustrates a perspective view of a corner of the face covering body showing a charging port;

FIG. 20 illustrates a side view of the face covering body including the back frame;

FIG. 2P illustrates a side view of the face covering body with the back frame removed.

FIG. 2Q illustrates a cross-sectional view of the face covering body including the back frame;

FIG. 2R illustrates a cross-sectional view of the face covering body with the back frame removed;

FIG. 2S illustrates a side view of the face covering body showing a charging cable inserted in the charging port;

FIG. 2T illustrates a bottom view of a portion of the face covering body showing the charging cable being inserted in the charging port;

FIG. 2U is an exploded view of the face covering having an outer frame, a back frame, and a front frame;

FIG. 3A illustrates an example embodiment of the face covering body having an inhale filtration subsystem and an exhale filtration subsystem;

FIG. 3B illustrates an example embodiment of the inhale filtration subsystem.

FIGS. 3C and 3D illustrate an example embodiment of an inhale filter cartridge configured to be received by one or more receptacles of the face covering body;

FIG. 3E illustrates an example fan that may be included in an air circulation subsystem of the face covering body;

FIG. 3F illustrates an example embodiment of the back frame of the face covering body having one or more inhale vent areas allowing the outside air to flow into a face area of the face covering body;

FIG. 3G illustrates an example embodiment of the front frame of the face covering body having one or more inhale vent areas allowing the outside air to flow into the face area of the face covering body;

FIG. 3H illustrates an example embodiment of the back frame of the face covering body having an exhale vent area;

FIG. 3I illustrates an enlarged view of a bottom area of the back frame of the face covering body having an exhaust filter attached thereto;

FIG. 3J illustrates a bottom view of the face covering body showing one or more exhaust slots disposed at the bottom of the front frame;

FIG. 3K illustrates an example embodiment of an intake filter;

FIG. 3L illustrates an example embodiment of an exhale filter;

FIG. 4A illustrates a front frame of the face covering body having an aperture for allowing outside sound to reach a microphone coupled to the back frame;

FIG. 4B illustrates a back frame of the face covering body having an aperture allowing sound from the face area to reach the microphone coupled to the back frame;

FIGS. 5A and 5B illustrate a right side perspective view and a left side perspective view of a front frame having a light subsystem glowing through the edge of puck(s);

FIG. 5C illustrates an example embodiment of the light subsystem having a center LED;

FIG. 5D illustrates an example embodiment of the light subsystem having an LED ring;

FIG. 6A illustrates a perspective view of the face covering body having a pair of earbuds attached thereto via cables;

FIG. 6B illustrates a back view of the face covering body having the pair of earbuds attached thereto via cables;

FIG. 6C illustrates a front view of the face covering having the pair of earbuds attached thereto via cables;

FIG. 6D illustrates an exploded view of an example earbud;

FIG. 6E illustrates different perspective views of the earbud of FIG. 6D;

FIG. 6F illustrates an example embodiment of a non-removable earbud having a wire connected to the face covering body;

FIG. 7 illustrates an example architecture of the smart mask;

FIG. 8 illustrates example control interface(s) that may allow a wearer to control or interface with the smart mask via voice commands, a terminal device, and/or a cloud service;

FIG. 9A illustrates a schematic diagram of an example system of the smart mask;

FIG. 9B illustrates a diagram of example inner connection(s) on a circuit board;

FIG. 9C illustrates an example flexible printed cables (FPC) connector having multiple pins;

FIG. 9D illustrates an example embodiment that a female connector part is on the circuit board, and a male connector part is on the FPC;

FIG. 9E illustrates a schematic diagram of the FPC that connects the circuit board(s);

FIG. 9F illustrates a schematic diagram of multiple control buttons on the PFC;

FIG. 9G illustrates an outline of an example printed circuit board (PCB) and its connectors;

FIG. 9H is a graph illustrating sensitivity of an example speaker or earphone at different frequencies;

FIG. 9I is a graph illustrating total harmonic distortion (THD) of an example speaker or earphone at different frequencies;

FIG. 9J illustrates schematic diagrams of example circuits for battery management and protection;

FIG. 9K illustrates schematic diagrams of example circuits for connecting and controlling the fan and LED via the FPC connector;

FIG. 9L illustrates an example battery cell that may be embedded in the smart mask;

FIGS. 9M and 9N illustrate both sides of an example printed circuit board (PCB) having various connectors;

FIG. 10A illustrates an example home page of a mobile application for controlling and interfacing with the smart mask;

FIG. 10B illustrates an example air control interface of the mobile application for controlling an air circulation subsystem of the smart mask;

FIG. 10C illustrates another example home page of a mobile application for controlling and interfacing with the smart mask; and

FIG. 11 illustrates an example computing system in which the principles described herein may be employed.

### DETAILED DESCRIPTION

The embodiments described herein are related to smart devices and protective respirators or masks (hereinafter referred to as smart masks) that are configured to protect a wearer from inhaling hazardous atmospheres.

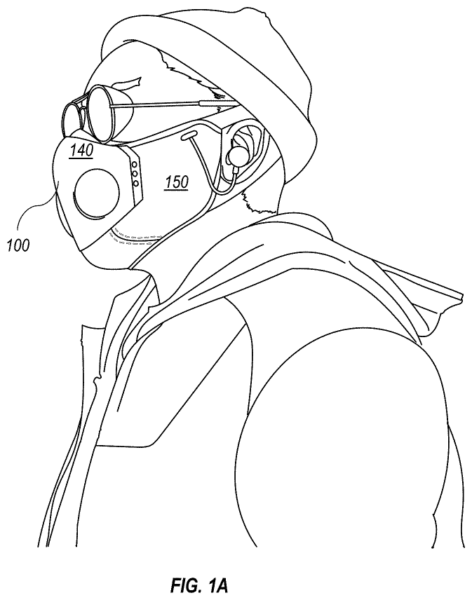

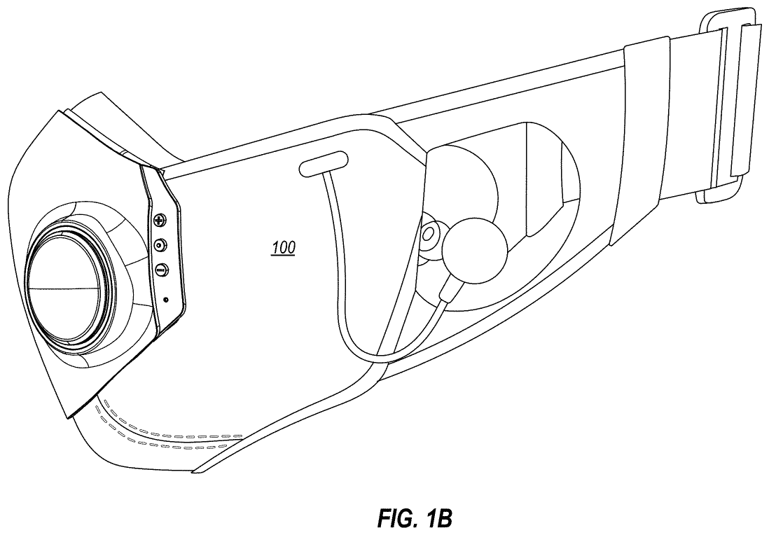

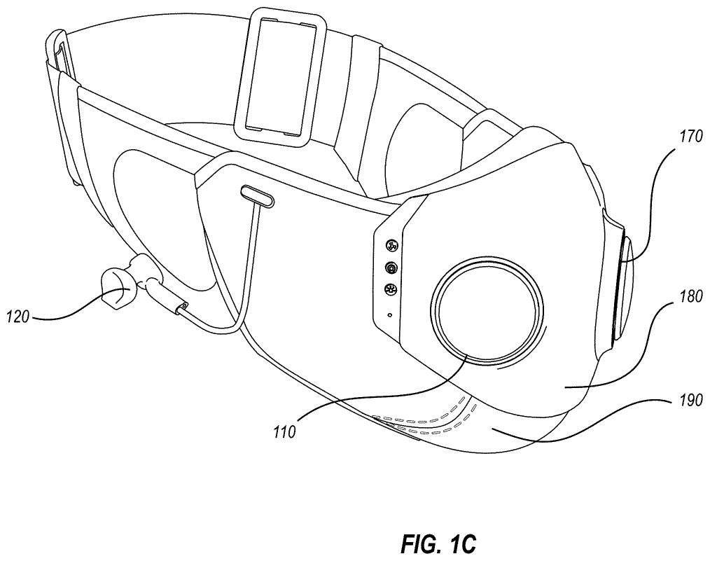

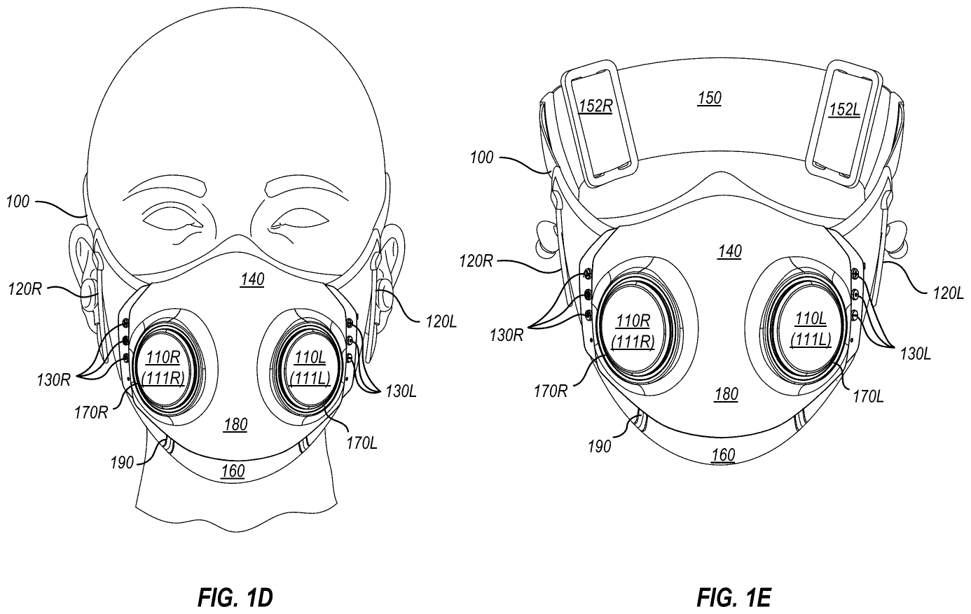

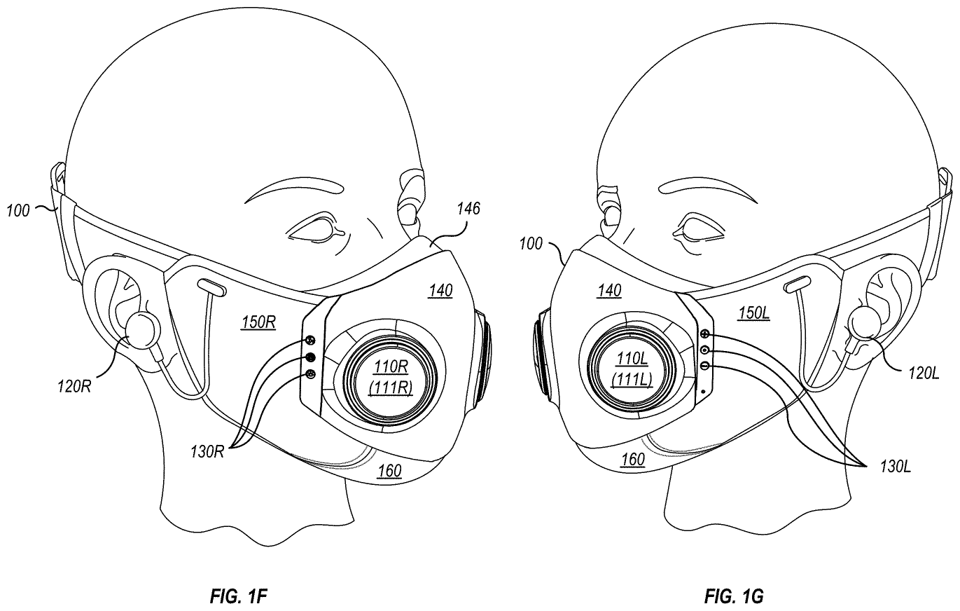

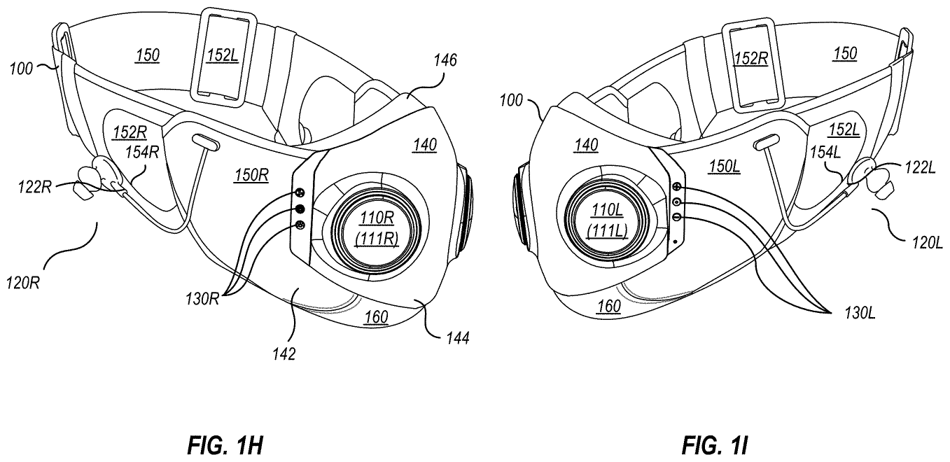

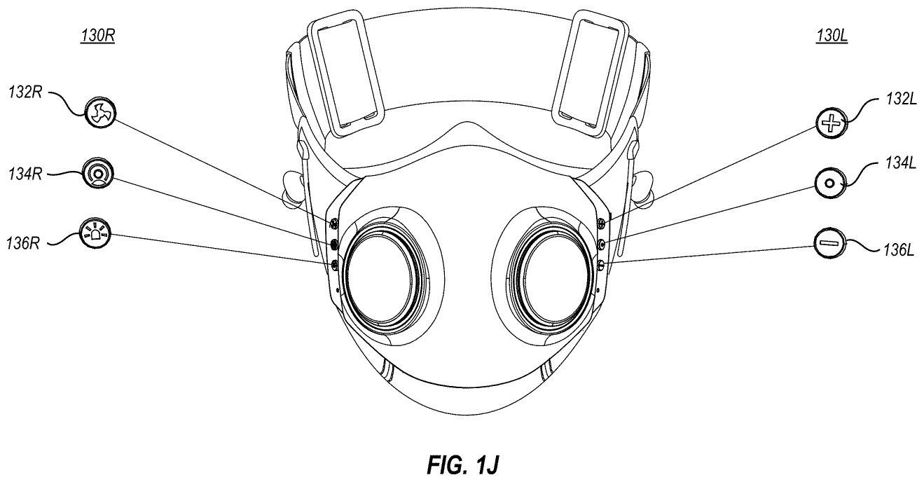

FIGS. 1A through 1J illustrate an example embodiment of the smart mask 100. FIG. 1A illustrates that the smart mask 100 is being worn by a wearer. FIG. 1B further illustrates a perspective view of the smart mask 100. As illustrated, the smart mask 100 includes a face covering 140 configured to cover the nose and mouth of the wearer when it is worn. The smart mask 100 also includes an attachment member 150 configured to attach the face covering 140 on the wearer's face. The attachment member 150 has an adjustable contour that ergonomically fits any wearer. The surface material(s) of the attachment member 150 and the face covering 140 is water-resistant, skin-friendly, and lightweight. In some embodiments, the attachment member 150 further includes two openings 152 that allow the wearer's ears to pass through and to receive earbuds 122. The earbuds 122 are parts of an audio subsystem of the smart mask 100 configured to be controlled by voice commands and/or by the physical controllers 130 disposed on the face covering 140.

In some embodiments, the structure of the mask is waterproof, e.g., IP54 waterproof. In some embodiments, the structure of the mask is also anti-drop, e.g., withstanding 1.5 m drop on marble floor without function and/or structure damage, and/or cosmetic condition meets specific standards.

Notably, the smart mask 100 is not only a piece of personal protective equipment, but also a piece of techwear. FIG. 1C further illustrates various technical components of the smart mask 100, including (but not limited to) an air circulation subsystem 110, a audio subsystem 120, a light subsystem 170, a noise reduction subsystem 180, and a power charging port 190. In some embodiments, the air circulation subsystem 110 includes an inhale filtration subsystem and an exhale filtration subsystem 160. The air circulation subsystem 110 includes one or more fans configured to draw outside air into the face covering. The inhale filtration subsystem includes an inhale filter or filter cartridge configured to filter the outside air before the outside air enters the face area inside the face covering 140. The exhaust subsystem 160 includes an exhale filter or filter cartridge configured to filter the exhale air before the exhale air exits the face covering 140. The audio subsystem 120 includes one or more speakers or one or more earbuds configured to receive audio signals from other computing devices and play the received audio signals to the wearer. The audio subsystem 120 may also include one or more microphones configured to receive user's voice, allowing the user to give voice commands, receive phone calls, or perform other functions that requires a microphone.

The noise reduction subsystem 180 includes a noise reduction microphone configured to receive surrounding noise (including, but not limited to, noise generated by the fans of the air circulation subsystem, or noise generated by vehicle, airplane, or any surrounding machine) and provide feedback or feedforward to the audio subsystem 120 to cause the audio subsystem 120 to emit a sound wave with the same amplitude of the surrounding noise but with inverted phase to the surrounding noise to cancel out the surrounding noise. The light subsystem 170 includes one or more LED light(s) that may be embedded anywhere on the face covering or the attachment member 150. As illustrated, in some embodiments, the light subsystem 170 is coupled to the air circulation subsystem 110.

In embodiments, the air circulation subsystem 110, the light subsystem 170, the noise reduction subsystem 180, and/or the audio subsystem 120 are powered via one or more rechargeable lithium batteries. A power charging port 190 is disposed at a lower side of the face covering 140 configured to charge the one or more rechargeable lithium batteries. Further, in embodiments, these electronic components can be continuously used while the batteries are being charged. Additional details about each of these components are further described below with respect to the rest of the figures.

FIGS. 1D and 1E illustrate a front view of the smart mask 100 when the smart mask 100 is worn or not worn by a wearer. FIGS. 1F and 1G illustrate a right side view and a left side view of the smart mask 100, when the smart mask 100 is worn by a wearer. FIGS. 1H and 1I illustrate a right side view and a left side view of the smart mask 100 when the smart mask 100 is not worn.

As illustrated in FIGS. 1D through 1I, the smart mask 100 includes a face covering 140, an attachment member 150, an air circulation subsystem 110R, 110L, an exhaust subsystem 160, an audio subsystem 120R, 120L, a light subsystem 170R, 170L, one or more controller(s) 130R, 130L, an ANC subsystem 180, and a charging port 190. The face covering 140 is configured to cover a face area of a wearer. The attachment member 150 is configured to attach the face covering 140 onto the face area of the wearer. The air circulation subsystem 110R, 110L is configured to draw outside air into the face area, such that the wearer can breathe easily, and the air in the face area is kept cool and dry. In embodiments, the air circulation subsystem 110R, 110L may further be coupled to an inhale filtration subsystem (which will be further described later with respect to FIGS. 3A-3G) configured to filter the outside air before the outside air is drawn into the face area of the face covering.

The exhaust subsystem 160 is configured to purge the exhaled air out of the face area. In some embodiments, the exhaust subsystem 160 may also be coupled to an exhale filtration subsystem (which will be further described later with respect to FIGS. 3H-3J) configured to filter the exhale air before releasing the exhale air out of the face covering. The ANC subsystem 180 includes an ANC microphone (which will be further described later with respect to FIGS. 4A and 4B) configured to receive surrounding noise and provide feedback or feedforward to the audio subsystem 120. The light subsystem 170R, 170L includes one or more LED lights (which will be further described later with respect to FIGS. 5A-5D) configured to create a dope flow effect around the air circulation subsystem 110R, 110L. The controller(s) 130R, 130L are configured to control the air circulation subsystem 110R, 110L, the audio subsystem 120R, 120L, and the ANC subsystem 180.

The air circulation subsystem 110R and 110L includes a right portion 110R placed on a right side of the face covering 140, and a left portion 110L placed on a left side of the face covering 140. The exhaust subsystem 160 is placed below the air circulation subsystem 110R, 110L. The audio subsystem 120R, 120L, includes a pair of earbuds 120R and 120L. The attachment member 150 may be an adjustable band, including one or more adjustable buckles 152R and 152L configured to adjust the size of the band.

Further, referring to FIGS. 1H and 1I, in some embodiments, each of the pair of earbuds 120R, 120L further includes a magnetic portion or a metal portion 122R or 122L, and the attachment member 150 also includes a pair of magnetic portions or metal portions 154R, 154L near the pair of earbuds on the right side 150R or left side 150L, such that when the earbuds are not worn, they are configured to be secured to the corresponding magnetic portion or metal portion 154R, 154L of the attachment member 150.

In some embodiments, each side of the air circulation subsystem 110R or 110L includes a fan configured to draw outside air into the face area of the face covering. In some embodiments, each side of the air circulation subsystem 110R or 110L further includes an inhale filtration subsystem, including (but not limited to) a filter receptacle, a filter, and a removable puck 111R or 111L. The filter receptacle is configured to receive a filter cartridge for filtering the outside air before the air is drawn into the face area. The filter cartridge may be designed to meet various standard ratings, such as N95, N99, or N100. The fan is configured to draw outside air through the corresponding filter cartridge into the face area. The fan not only can help a wearer to breathe easily, but also prevent moisture and heat from building up in the face area. The removable puck 111R or 111L is configured to cover the corresponding filter cartridge or fan.

As illustrated in FIGS. 1D through 1I, the smart mask 100 further includes one or more controllers 130R and 130L. FIG. 1J further illustrates an enlarged view of the right controller(s) 130R and the left controller(s) 130L of the smart mask 100. In embodiments, one or more right controllers (e.g., buttons) 130R are positioned on the right side of the face covering, and one or more left controllers (e.g., buttons) 130L are positioned on the left side of the face covering 140. Referring to FIG. 1J, the left controller(s) 130L is configured to control the audio subsystem 120R, 120L. In embodiments, the left controller(s) 130L includes a volume up button 132L, a volume down button 136L, and a center button 134L. A wearer may use the volume up button 132L or volume down button 136L to control the volume of the audio subsystem. A wearer may use the center button 134L to pause or play a song or audio file. A wearer may also use the controllers 132L, 134L, and/or 136L to fast-forward, rewind, and/or skip a song or audio file, by pressing or tapping the controllers 132L, 134L, and/or 136L once, twice, three times, and/or long-pressing, etc.

On the other side, the right controller (s)130R are configured to control the air circulation subsystem 110, the active noise cancellation (ANC) subsystem 180, and the light subsystem 170. In embodiments, the controller 130R includes a fan button 132R, an ANC button 134R, and a light button 136R. A wearer may use the fan button 132R to turn on or off the air circulation subsystem 110, use the ANC button 134R to turn the ANC subsystem on or off, and use the light button 136R to turn on or off the light subsystem. In some embodiments, the air circulation 110 includes one or more fan(s), which may be configured to operate at different speeds (e.g., low, medium, high, etc.), and the fan button 132L may further be used to control the speed of the fan(s). In some embodiments, the light subsystem may include one or more light source(s) configured to emit a light beam at different intensities, and the light button 134L may further be used to control the intensities of the light.

As illustrated in FIGS. 1A-1J, the face covering 140 of the smart mask 100 includes a body which is at least partially covered by water resistant, skin-friendly, and lightweight material or fabric. Inside the fabric, various structural elements may be implemented to allow the above-described technical components to be embedded in the body of the face covering 100. In some embodiments, the body of face covering may include a front frame configured to receive and secure various technical components, a back frame, and an outer frame configured to receive the back frame. The front side is a side exposed to the outside when the smart mask 100 is worn by the user. The back side is a side that covers a user's face when the smart mask 100 is worn by the user.

FIGS. 2A-2G illustrate various back side views of exemplary structural elements of a body 200 of a face covering of a smart mask (hereinafter also referred to as the “face covering body”). FIGS. 2H-2K illustrate various front side views of exemplary structural elements of the face covering body 200.

FIGS. 2A and 2D illustrate a back side of the face covering body 200, showing an outer frame 210 and a back frame 230. The outer frame 210 constitutes an outer edge of the face covering body 200. The back frame 230 is mounted on the outer frame 210. The outer frame 210 and the back frame 230 are configured to cover a face of a user. The back frame 230 includes a center area 222, a left fan area 224, a right fan area 226, and a bottom exhale area 228. The center area 222 has a concave portion that has a contour similar to users' nose. The left and right fan areas 224 and 226 are substantially planar. The direction and angle of the left and right planar fan areas 224, 226 are configured to fit to a shape of users' face. Further, each of the left and right fan areas 224, 226 also has a vent area that includes one or more apertures to allow inhale air to come in. The bottom exhale area 228 also has an opening configured to allow exhale air to pass out. In embodiments, the back frame 230 is mounted on the outer frame 210 via snap-on latches and/or one or more screws.

FIGS. 2B, 2C, and 2E further illustrates views of the face covering body 200 when the back frame 230 is removed. When the back frame 230 is removed, a back side of a front frame 250 is shown. Various electronic components are housed between the front frame 250 and the back frame 230. As illustrated in FIGS. 2B, 2C, and 2E, one or more circuit board(s) 242, 244, one or more fans 252, 254, and an exhale filter 260 of the exhaust subsystem are disposed between the front frame 250 and the back frame 230. The circuit board(s) 242, 244 may be a printed circuit board (PCB) configured to support and electrically connect the different electronic components using conductive tracks, pads, and other features. One or more processor(s) may also be coupled to the circuit board(s) 242, 244 to control the different electronic components. Further, the circuit board(s) 242, 244 may also be coupled to a rechargeable battery 247. These rechargeable batteries 247 may be 5-volt batteries configured to power the different electronic components, such as the fan(s) 224, 227, the earbuds or speakers, the microphone(s), and the processor(s).

Referring to FIG. 2C, each fan 252, 254 is configured to be inserted and secured in the corresponding opening of the front frame 250. FIGS. 2F and 2G illustrate enlarged perspective views of the back side of the front frame 250. A charging port 246 is connected to the right-side circuit board 244 configured to charge the batteries 247 coupled to the circuit board 242, 244. The charging port 246 may be any standard port, including (but not limited to) a USBC port, a micro USB port.

Referring to FIG. 2E, the back side of the front frame 250 also has an aperture coupled to an ANC microphone 249. The ANC microphone 249 may be placed on or next to the circuit board 242 and/or the fan 252. The ANC microphone may also be powered by the batteries 247 coupled to the circuit board 242. The ANC microphone is configured to detect various noise, such as the noise generated by the fan, and send the detected noise to the audio subsystem via the circuit board 242. 244.

In some embodiments, the front frame 250 is further coupled to one or more magnetic portions 248, and the frame of the filter cartridge 282, 284 includes a metal portion configured to be attracted secured by the magnetic portion(s) 248.

FIGS. 2H-2K illustrate different front side views of structural elements of the face covering 200. FIGS. 2H-2J illustrate a front side of the front frame 250. As illustrated, a top portion of outer frame 210 is attached to the front frame 250. The front frame 250 includes a center portion 253, a left portion 255, a right portion 256, a left edge 258, and a right edge 259. At each of the left edge portion 258 and 259, there are a plurality of small openings, each of which is configured to allow a control button 130R, 130L connected to the circuit board 242, 244 to pass through.

Referring to FIGS. 2I and 2J, on each of the left portion 254 and right portion 256, there is a larger opening having a bracket 292, 294 attached thereto. The bracket 292. 294 may constitute a filter receptacle configured to receive an inhale filter cartridge 282, 284. At the same time, the bracket 292, 294 may also be a support portion for the light subsystem configured to hold one or more LED lights. In embodiments, each bracket 292. 294 is circular shaped, and an LED light 296, 298 is mounted at the center of the bracket 292, 294. Alternatively, in some embodiments, multiple LED lights are disposed along the outer edge of the frame to form a ring. The inhale filter cartridge 280 is annular shaped configured to fit the bracket 292, 294. The hollow center and/or the outer edge of the annular inhale filter cartridge 282, 284 allows the LED light 296, 298 to pass through. The pucks 272, 274 are configured to cover and hold the corresponding filter cartridge 282, 284 in place.

Further, to allow air to flow into the inhale filter cartridge 282, 284, each puck 272, 274 is configured to be secured at a position that is slightly above the edge of the bracket 292, 294 (and/or the edge of the opening of the outer frame 210), such that a gap is formed between each puck 272, 274 and the edge of the corresponding opening of the outer frame 210. The gap also allows the LED light 296, 298 to shine through to create a dope glow effect. FIGS. 2I-2J further illustrate that each of the puck 272, 274 is removably attached on the edge of the opening of the outer frame 210. In embodiments, each puck 272, 274 may have a plurality of snap-on latches or serrated edges configured to engage with the edge of the opening of the outer frame 210 or the bracket 290.

Referring to FIG. 2E, in some embodiments, a magnetic component 248 is included on each side of the circuit board 242, 244, or the front frame 250. The position of the magnetic component 248 is near and/or under the bracket 292, 294 of FIGS. 2I, 2J. In embodiments, the filter cartridge 282, 284 may include a metal frame configured to be attracted by the magnetic component 248 placed under the bracket 292, 294 to help secure the filter cartridge 282, 284 in place.

As illustrated in FIG. 2K, the various electronic components are disposed between the front frame 250 and the back frame 230. When the front frame 250 is removed, the various electronic components and the back frame 230 would be exposed. As illustrated, one or more fans 252, 254, the circuit board, and a plurality of control buttons are exposed. A charging cable is inserted into the charging port configured to charge the batteries coupled to the circuit board. Various electrical cords, conductive tracks 257, and/or pads are configured to connect the batteries, the circuit board, the fans 252, 254, the ANC microphone, the audio subsystem, and/or control buttons to each other.

FIGS. 2L-2T further illustrate perspective views of various components of the face covering body 200. FIGS. 2L, 20, and 2Q illustrate different perspective views of the back frame 230 of the face covering body 200. FIGS. 2M, 2P, and 2R illustrate different perspective views of the face covering body 200 when the back frame 230 is removed. FIGS. 2N, 2S, and 2T illustrate different perspective views of different portions of the face covering body 200 near the charging port. As illustrated, each fan is mounted under the corresponding light frame, filter cartridge, and puck. When the fan is turned on, the outside air is drawn through the filter cartridge into the interior side of the face covering body 200. On the other side, the exhaled air passes through the exhaust subsystem to the front side of the mask.

FIG. 2U illustrates an exploded view of the face covering body 200, including an outer frame 210, a back frame 230, and a front frame 250. The back frame 230 is placed between the outer frame 210 and the front frame 250. The front frame 250 is configured to receive the inhale filtration subsystem and the LED light subsystem. The fan(s), printed circuit board(s), and an exhale filtration subsystem are housed between the front frame 250 and the back frame 230 and coupled to the back frame 230.

Turning now to FIGS. 3A-3J, the illustrated are the air circulation subsystem 310 (corresponding to the air circulation subsystem 110 of FIGS. 1D-1I), including the inhale filtration subsystem and the exhaust subsystem 320 (corresponding to the exhaust subsystem 160 of FIGS. 1D-1I) in more details. Referring to FIG. 3A, the inhale filtration subsystem 310 includes an inhale filter or filtering cartridge configured to filter the outside air before the outside air enters the face area of the face covering; and the exhale subsystem 320 includes an exhale filter or filtering cartridge configured to filter the exhale air before the exhale air exits the face covering. The inhale filter corresponds to the filter 282, 284 of FIGS. 2I and 2J. The exhale filter corresponds to the filter 260 of FIGS. 2B-2C and 2E. In some embodiments, the inhale filter(s) for the inhale filtration subsystem 310 is a triple-layered antimicrobial antiviral copper-woven DEKA Fab filter configured to kill 99.9% of bacteria.

FIG. 3B illustrates an exemplary embodiment of the inhale filtration subsystem 310 configured to be embedded in an opening of the front frame 230 of the face covering body 200. In embodiments, the inhale filtration subsystem 310 includes a bracket 312 (corresponding to the bracket 292, 294 of FIGS. 2I and 2J), a filter cartridge 314 (corresponding to the filter cartridge 282, 284 of FIGS. 2I and 2J), and a puck 316 (corresponding to the puck 272, 274 of FIGS. 2I and 2J). The bracket 312 is a circular shaped bracket having a protrusion portion at the center. The filter 314 is an annular shaped filter configured to fit on the bracket 312. The protrusion portion of the bracket 312 is configured to pass through the hollow center of the annular shaped filter 314 to secure the filter 314 in place. The puck 316 is configured to hover over the filter 314, i.e., substantially cover the filter but leave a gap between the filter 314 and the puck 316 to allow inhale air to flow through.

In some embodiments, the protrusion portion of the bracket 312 further includes an LED light 318 (which corresponds to the LED light 296, 298 of FIGS. 2I-2J). The LED light 318 is configured to shine through the gap between the puck and the filter to create a dope glow effect. In some embodiments, the puck 316 may be transparent or semi-transparent, such that the LED light 318 may at least partially shine through the puck 316. In some embodiments, multiple LED lights are evenly disposed along the outer edge of the frame 312 to form a ring, and the ring of the LED lights is configured to shine through the edge of the puck 316.

FIG. 3C further illustrates a outer frame 210 of the face covering body 200, on which the pucks 316 are removed. When the pucks 316 are removed, the filter(s) 314 (314R or 314L) is exposed to a user, and the user can easily replace the filters 314 if needed. In some embodiments, each bracket or the corresponding area of the back frame further includes a magnetic portion (e.g., magnetic portion 248 of FIG. 2E), and each filter cartridge 314 includes a metal frame 313 configured to be attracted by the magnetic portion, as illustrated in FIG. 3D.

The filter cartridge 314 may be made from various materials including (but not limited to) (1) an FDA-approved antimicrobial DEKA Fab filtration material, (2) an FDA-approved DEKA Fab copper-woven Cupron fibers, (3) polypropene infused copper oxide DEKA fab filter, (4) active antiviral filtration material, (5) antimicrobial filtration material, (6) uniquely antimicrobial and antiviral filtration material, and/or (7) high-quality breathable filtration material. In some embodiments, the filter cartridge 314 is configured to eliminate 99.9% of bacterial and/or 99.9% of all aerosol-based pathogens. In some embodiments, the filter cartridge 314 has a bacterial filtration efficiency (BEF) greater than 98%. In some embodiments, the filter cartridge 314 has a sub-micron particulate efficiency (PFE) at 0.1 um and/or greater than 98%. In some embodiments, the filter cartridge 314 has a breathability of 122 mm Hg. In some embodiments, the filter cartridge 314 may be washable and/or replaceable.

The inhale filtration subsystem 310 is configured to filter the outside air drawn into the face area of the face covering body 200 by the air circulation subsystem. FIGS. 3E-3G further illustrate the air circulation subsystem in detail. The air circulation subsystem includes one or more fans 322, corresponding to the fans 252, 254 of FIGS. 2B, 2C. The fan 322 includes a nose 324, a plurality of blades 325, and a mounting frame 323. In some embodiments, the fan 322 may be configured to run at multiple speeds. In some embodiments, the fan 322 may have a quiet mode, in which the ANC subsystem is automatically turned on. The fan 322 is configured to be mounted on the back frame 230, the circuit board 242, 244, or a separate frame structure coupled to the back frame 230.

The fan may have a dimension of 25×25×6 mm. the fan may be rated as about 5 volts and 13000 RPM±15% at rated voltage. The air flow of the fan may be about 3.0 CFM, and the acoustic noise of the fan may be about 24.8 dB(A).

The air circulation subsystem also includes one or more inhale vent areas on each of the outer frame 210 and back frame 230 of the face covering 200 to allow the air to flow through. FIGS. 3F and 3G further illustrate a back frame 230 and a front frame 250 of the face covering body 200. Referring to FIG. 3F, the back frame 230 includes two inhale vent areas 326, each of which having a plurality of openings corresponding to the shape of the fan blades. Referring to FIG. 3G, the front frame 250 also includes two inhale vent areas, each of which having a plurality of openings corresponding to the shape of the fan blades. In particular, each of the inhale vent area 326 or 328 is disposed at each of the left side and right side of the back frame 230 or the front frame 250 corresponding to the area where the fan 252, 254 is disposed. Each of the inhale vent areas 326, 328 is configured to allow the air blown by the fan(s) 322 to flow through from the outside of the face covering body 200 into the face area of the face covering body 200.

The face covering body 200 also includes an exhaust subsystem having an exhale filtration subsystem. FIGS. 2H-2J illustrate the exhaust subsystem and the exhale filtration subsystem in further details. FIG. 3H illustrates a back frame 230 of the face covering body 200, and an exhale vent area 332 is disposed at a lower portion of the back frame 230. The exhale vent 332 allows the exhale air to pass through. FIG. 3I illustrates a back frame 230 of the face covering body 200 having an opening, and an exhaust filter or filter cartridge 324 is disposed at the opening. The exhaust filter or filter cartridge 324 is configured to filter the exhale air before the exhale air exists the face covering body 200. As illustrated, the exhaust filter 324 may be a DEKA FAB filter having a dimension of 26 mm×26 mm. FIG. 3J illustrates a bottom view of the face covering body 200. One or more slot-shaped filter exhausts or vents 326 are disposed at a bottom area of the outer frame 210. The filter exhausts 326 of the outer frame 210 are configured to let the filtered exhale air to exit the face covering body 200.

In embodiments, the inhale and exhale filtration subsystems include two intake filters and one exhaust filter. Each of the two intake filters and the exhaust filter may be made from HEPA grade filter pleated material to maximize air intake and exhaust efficiency. FIG. 3K further illustrates another example embodiment of intake filter. In some embodiments, an outer diameter of the intake filter is about 42 mm, an inner diameter of the intake filter is about 10 mm, and a thickness of the intake filter is about 9 mm. In some embodiments, the intake filter is made from H12 HEPA having a BEF level greater than 98. The sealing method of the intake filter may include thermoplastic elastomer TPE overmolding with 2 mm thickness. The breathing filter area is about 40-50 cm².

FIG. 3L illustrates another example embodiment of exhaust filter. In embodiments, the exhaust filter has a size of about 22 mm long, 10 mm wide, and 12 mm height. The exhaust filter may also be made from H12 HEPA having BEF level greater than 98. In embodiments, the sealing method may be thermoplastic elastomer (TPE) overmolding with 1 mm to 1.5 mm thickness; and the breathing filter area is about 10-15 cm².

As previously discussed, the face covering body 200 also includes an ANC subsystem configured to automatically cancel certain noise (e.g., the noise generated by the fan). The ANC subsystem includes an ANC microphone 249 coupled to the back frame 230 or the circuit board 242, 244. FIG. 4A illustrates a front side of the back frame 230 of the face covering body 200, having an aperture 410 configured to allow sound (generated from the outside of the face covering body 200, such as ambient noise) to pass through and reach the microphone 249 (see FIG. 2E) coupled to the back frame 230. FIG. 4B illustrates the back side of the back frame 230 of the face covering body 200, also having an aperture 420 configured to allow sound (from the face area of the face covering body 200, such as voice command) to pass through to reach the microphone 249 coupled to the back frame 230 (see FIG. 2E).

In some embodiments, more than one microphone is implemented in the smart mask, and the microphones 249 are not only configured to receive the noise generated inside and outside of the smart mask, but also configured to receive voice of the wearer. For example, the noice reduction system allows the wearer's voice during phone calls to be more clear and/or eliminates mask-muffled phone calls. In some embodiments, two electret condenser microphone (ECM) miniature condenser microphones are implemented, and each of which is placed in one side of the back frame 230 of the face covering body 200. In some embodiments, one or more micro-electromechanical systems (MEMS) microphone(s) are implemented as part of the noise reduction subsystem. In some embodiments, a Qualcomm® cVc audio 8^{th} generation technology is implemented. In some embodiments, Qualcomm® aptX™ is supported. In some embodiments, quad-core processing, dual core 32-bit processor application subsystem, a dual core Qualcomm® Kalimba™ Digital Signal Processing (DSP) Audio subsystem, and/or processors having extremely low power design may be implemented. In some embodiments, an embedded ROM, RAM, and/or external Q-SPI Flash memory is used in the audio subsystem and/or the ANC subsystem. In some embodiments, the ANC subsystem is a comprehensively programmable digital ANC subsystem designed for reduced eBoM through highly integrated SoC design.

Also, as previously discussed, the face covering body 200 may include one or more light subsystems. FIGS. 5A-5C illustrate example embodiments of the light subsystems. As illustrated, a light source is placed under each puck 510 (corresponding to the puck 272, 274 of FIG. 2I, 2J) and configured to shine through the edge 520 of the puck 510. Referring to FIG. 5C, when the puck 510 is removed, a bracket 540 (corresponding to the bracket 292, 294 of FIGS. 2I-2J) and the light source 530 (corresponding to the LED light 296, 298) are exposed to the user. The light source 530 may be a LED light configured to be attached to a center of the bracket 540. The LED light may be configured to emit a light of different colors, wavelengths, patterns, and/or intensities. A user may be allowed to control the color, wavelengths, pattern, and/or intensity of the light via the control button 136R. In some embodiments, the puck 510 may also be transparent or semi-transparent, such that the light emitted by the light source 530 may also at least partially shine through the puck 510. In some embodiments, the light may be configured to indicate the on/off or speed status of the fan(s). In some embodiments, the light may also be configured to indicate the on/off status of the audio subsystem. In yet some embodiments, the light may also be configured to indicate the power level of the battery and to prompt the user to charge the smart mask 100 when the power level is low.

In some embodiments, the light may be an LED ring. FIG. 5D illustrates an example LED ring, along which there are a plurality of LED lights. For example, in some embodiments, 12 pieces of 1.6×0.8×0.6 mm LEDs may be evenly distributed on a circular shaped printed circuit board (PCB).

In some embodiments, the LED lights may have different daytime and/or nighttime glow power settings. In some embodiments, the LED lights may be configured to create dope glow effect, and/or woo woo light effect.

Additionally, in some embodiments, the light subsystem may further include one or more ultraviolet LED light configured to emit ultraviolet light for sanitizing the filter(s), the fan(s), and the various surfaces of the components of the face covering body 200.

Finally, the smart mask 100 also includes an audio subsystem. The audio subsystem may include a pair of earbuds. FIGS. 6A-6C illustrates an example embodiment of the audio subsystem 600, which may be a pair of built-in high sound quality Bluetooth earbuds 612, 614 that are coupled to the face covering body 200. In some embodiments, each of the earbuds 612, 614 may be configured to be removably secured at a magnetic docking station 622, 624 that is coupled to the attachment member 150 of the smart mask 100. In some embodiments, the docking station 622, 624 may include contact connections for charging the earbud 612, 614. Alternatively, the earbuds are connected to the batteries inside the face covering via cables.

Further, the earbuds 612, 614 may also be configured to receive signals from the ANC microphone to generate a sound wave with the same amplitude but with inverted phase to the surrounding noise to cancel out the surrounding noise. In some embodiments, the earbuds 612, 614 are voice assistant ready. In some embodiments, a single tap at a control button (or any control buttons) would allow the user to accept phone calls. In some embodiments, a user can also turn on and off noise cancellation mode by pressing the ANC button. In some embodiments, the earbuds are configured to receive audio boom sounds via Bluetooth 5.0 connectivity.

FIG. 6D further illustrate an exploded view of an example earbud 612. The earbud 612 includes an ear tip 631, an ear pad 632, a cap 633 a cone 634, an ear cup 635, and a disk 636. The ear pad 632 may include a dust film preventing dust from getting into the interior of the earbud. Between the cap 633 and the disk 636, there may be various electronic components, including (but not limited to) a micro driver, a printed circuit board, a processor, a battery, and/or an antenna.

FIG. 6E further illustrates two perspective views of the earbud 612 of FIG. 6D. The ear tip 631 may have a plurality of different sizes, such as small, medium, and large. The ear tip 631 may be made from silicone, having VDI18 mold texture. The cap 633, the ear cup 635, the cone 634, and the disc 636 may be made from Acrylonitrile Butadiene Styrene (ABS), having VDI18 mold texture. There may be an acoustics mesh coupled to the cap 634 and under the ear tip 631. In some embodiments, the earbud 612 also has a cable 637 configured to connect the earbud 612 to the audio subsystem's printed circuit board inside the mask. In some embodiments, the cable is about 2.2 mm diameter, wrapped by fabric.

In some embodiments, the two earbuds are designed as non-detachable. As illustrated in FIG. 6F, each of the earbuds includes a cable connected to the audio subsystem board. The length of the cable may have a few predetermined sizes, such as small/medium size of 120 mm, medium/large size of 140 mm.

Note, even though the embodiments illustrated in the figures use rechargeable batteries, it is not necessary that rechargeable batteries are required. In some embodiments, the smart mask 100 may be configured to be powered by disposable batteries. Also, there may be any number of batteries embedded in the face covering body 200. For example, a separate battery may be coupled to each of the left side and/or right side of the face covering 200.

Further, in some embodiments, a temperature sensor may be coupled to at least one of the earbuds 120 to detect an inner-ear temperature of the wearer. In some embodiments, a heart rate monitor may be coupled to the attachment member 150 to detect a heart rate of the wearer. In some embodiments, the network interface(s) is configured to connect to a user terminal (e.g., a mobile device) and/or a cloud service to transmit status data and/or grant control of the smart mask 100 to the user terminal. For example, the user terminal may be required to install a mobile app associated with the smart mask 100. After installing the mobile app, the user terminal is allowed to receive various status data and sensor data from the smart mask 100, and the user terminal is also able to control the smart mask 100 via the mobile app, such as turn on or off the air circulation subsystem, turn on or off the light sources, and/or turn on or off the audio subsystem.

FIG. 7 illustrates an example architecture 700 of the smart mask, which corresponds to the smart mask 100 of FIGS. 1A through 6D. As illustrated in FIG. 7, the smart mask 700 includes a face covering 740, an attachment member 730, an air circulation subsystem 710, an inhale filtration subsystem 714, an exhaust subsystem 720, a audio subsystem 770, an ANC subsystem 774, a light subsystem 780, one or more power source(s) 760, and one or more controller(s) 750. The face covering 740 is configured to cover a face area of a wearer. The attachment member 730 is configured to attach the face covering 740 onto the face area of the wearer. The air circulation subsystem 710 is powered by at least one of the one or more power source(s) 760 and configured to filter outside air and draw the filtered outside air into the face area. The exhaust subsystem 720 is placed below the air circulation subsystem and configured to purge exhaled air out of the face area. The one or more controller(s) 750 is configured to control the smart mask 700, including but not limited to the air circulation subsystem 710, the audio subsystem 770, the ANC subsystem 774, and the light subsystem 780.

The inhale filtration subsystem 714 includes one or more vent areas in each of the front frame and back frame and a filter cartridge placed in the air path between the vent areas of the front frame and back frame to filter the air flowing through the vent areas.

In embodiments, the air circulation subsystem 710 includes an inhale filtration subsystem 714 that includes one or more fan(s), which may be configured to run at different speeds. Each of the one or more fan(s) is powered by at least one of the one or more power source(s) 760 and configured to draw outside air through the inhale filtration subsystem into the face area.

In some embodiments, the one or more fan(s) includes a left fan placed on a left side of the face area and a right fan placed on a right side of the face area. The inhale filtration subsystem 714 includes a left filter cartridge and a right filter cartridge, each of which corresponds to the left fan and the right fan. In some embodiments, the inhale filtration subsystem 714 also includes one or more filter receptacle(s) (each of which is configured to receive a filter cartridge, and one of more puck(s) (each of which is configured to substantially cover a corresponding filter cartridge). For example, in embodiments, the one or more puck(s) includes a left puck and a right puck, and the one or more filter receptacles includes a left receptacle and a right receptacle. The left receptacle is configured to receive the left filter cartridge, and the right receptacle is configured to receive the right filter cartridge. The left puck is configured to cover the left filter cartridge, and the right puck is configured to cover the right filter cartridge. In some embodiments, each of the one or more filter receptacle(s) may further include a magnetic portion configured to hold a metal frame of a filter cartridge in place magnetically. In some embodiments, each of the one or more filter receptacle has a circular-shaped recess configured to receive a circular-shaped filter cartridge. In some embodiments, each of the one or more fan(s) is configured to operate at different speeds, and the controller 250 is configured to control a speed of the one or more fan(s).

In some embodiments, the air circulation system 710 also includes an exhale filtration subsystem 716 configured to filter the exhale air before the exhale air exits the face covering 740

In some embodiments, the smart mask 200 also includes an audio subsystem 870. The audio subsystem 770 is also powered by at least one of the one or more power source(s) 760 and configured to receive a sound signal wirelessly from a user terminal. The controller 750 is further configured to control the audio subsystem 770. In some embodiments, the audio subsystem 770 includes one or more speakers (e.g., a pair of speakers) or one or more earbuds (e.g., a pair of earbuds) 772. In some embodiments, the one or more speakers or earbuds 772 are Bluetooth speakers or Bluetooth earbuds that are configured to receive sound signals from a user terminal via BLE beacons. In some embodiments, each of the earbuds 772 includes a magnetic portion or a metal portion, and the attachment member 730 also includes a pair of magnetic portions or metal portions corresponding to the pair of earbuds, such that when the pair of earbuds are not in use, each of the pair of earbuds is configured to be secured to the corresponding magnetic portion or metal portion of the attachment member 730 magnetically.

In some embodiments, the audio subsystem 770 also includes an ANC subsystem 774. The ANC subsystem 774 includes an ANC microphone configured to receive voice inputs and/or surrounding noise. The surrounding noise is used to generate feedback or feedforward to the audio subsystem 770, such that the audio subsystem 770 can emit a sound wave with the same amplitude but with inverted phase to the surrounding noise to cancel out the surrounding noise. The one or more controller(s) 750 is further configured to turn on and off the ANC subsystem 774. In some embodiments, when the ANC subsystem 774 is turned off, the microphone 776 can still be used as a regular microphone configured to receive and process voice command(s) from a user. In some embodiments, more than one microphone is implemented in the audio subsystem 770. At least one of the microphones is part of the ANC subsystem 774, and at least another one of the microphones is used for receiving voice from users.

In some embodiments, the smart mask 700 further includes a light subsystem 780. The light subsystem 780 may include one or more ultraviolet (UV) light source(s) 782 (e.g., one or more UV LEDs) and/or indicator light source(s) 782 (e.g., one or more white or color LEDs). The UV light source(s) 782 and the indicator light source(s) 784 are also powered by at least one of the one or more power source(s) 760. Each of the one or more UV light source(s) 780 and/or the indicator light source(s) 782 may be coupled to a corresponding one of the one or more puck(s), a corresponding one of the one or more filter receptacle(s), and/or a controller. In some embodiments, one or more ultraviolet light(s) 782 is positioned around each filter. The UV light source(s) 782 is configured to disinfect the face covering, the filter receptacle, the puck and/or an area around the filter cartridge. The indicator light source(s) 782 may be configured to indicate whether the corresponding fan is on or off, or merely to cause the corresponding puck to glow.

In some embodiments, the smart mask 700 may also include a temperature sensor 790 and/or a heart rate monitor 792. The temperature sensor 790 may be coupled to at least one of the earbud(s) 772 to detect the inner-ear temperature of a wearer. The heart rate monitor 792 may be coupled to the attachment member 730 to detect a heart rate of the wearer.

In some embodiments, the one or more power source(s) 760 are one or more batteries. For example, each of the earbud(s) may be powered by a separate set of batteries, operating at around 3-4 volts DC power, and each of the fan(s) may be powered by a separate set of batteries, operating at around 5 volts DC power. In some embodiments, the one or more batteries are rechargeable batteries, and the smart mask 800 also includes one or more charging port(s) 762 (e.g., USBC port, micro USB port) for charging the rechargeable batteries.

In some embodiments, some of the controller(s) 750 may be as simple as a power switch. In some embodiments, the controller(s) 750 may be a computing system that includes one or more processor(s) 752, one or more network interface(s) 754, and one or more control interface(s) 756. A control interface is an interface 756 that allows a user or wearer to interact with the various components of the smart mask 700.

FIG. 8 further illustrates an example control interface(s) 800 that corresponds to the control interface(s) 756 of FIG. 7. As illustrated in FIG. 8, the control interface(s) 800 includes controller buttons 310, which correspond to the controllers 130R and 130L illustrated in FIGS. 1A through 1F and FIGS. 1J and 1K. These controller buttons 810 may include one or more sound controller(s) 812, one or more fan controller(s) 814, one or more light controller(s) 816, and one or more ANC controller(s) 818. When the smart mask 100, 700 includes additional components (e.g., a microphone 776, a heart rate monitor 792, a temperature sensor 790, etc.), additional contact controllers may be implemented to control these additional components.

In some embodiments, the control interface(s) 800 may also include a voice control 820 that is configured to receive and process voice commands from a wearer. In some embodiments, the voice control 820 may simply be able to recognize a few pre-programmed commands, such as “turn on the fan”, “turn on the light”, etc. In some embodiments, the voice control 820 may include a smart AI component that is configured to process natural language voice commands and perform complex tasks based on the processed natural language voice commands. The control interface(s) 800 may also use wireless interface(s) to communicate with a cloud service 860 and/or a mobile application installed on a user terminal 850 to allow a user to control the smart mask 100, 700 via the mobile application.

FIG. 9A illustrates a schematic diagram of an example system of the smart mask 100, 700. The Bluetooth audio subsystem of FIG. 9A corresponds to the audio system illustrated in FIGS. 6A-6G. In embodiments, the Bluetooth audio subsystem provides full Bluetooth earphone functions to the mask. Users may place phone calls clearly without removing the mask. Meanwhile, users can enjoy high-quality music while wearing a mask. In embodiments, the Bluetooth audio subsystem may include a computer processor having various build-in feed-forward ANC features.

In particular, the processor is designed to meet the demand for robust, high quality, wireless Bluetooth listening experiences in small devices with low power consumption for longer audio playback. In some embodiments, the processor is also designed to support voice assistants through cloud services. In some embodiments, the processor also features digital active noise canceling (ANC) technology integrated in the system on the chip (SoC) to eliminate the need for an external ANC solution.

In some embodiments, the Bluetooth audio subsystem (also referred to as the audio subsystem or the earphone subsystem) shares the main battery with the mask subsystem (including the air circulation subsystem and the light subsystem). In some embodiments, the audio subsystem directly takes power from the battery cell, and keeps monitoring the battery level. In some embodiments, the audio subsystem is also configured to send the battery level to the user's mobile terminal (e.g., a mobile phone), allowing the mobile terminal to display the battery level to the user. In some embodiments, when the battery level is lower than a predetermined threshold(e.g., 20%), the audio subsystem generates an alarm. The alarm may be a voice notification and/or a light notification (e.g., having at least one of the LED lights flashes red).

In some embodiments, the internal structure of the mask is designed to have the left side battery, fan, and LED ring connect to the audio system board, which, in turn, connects the fan controller and power board located at the right side of the mask through flexible printed cables (FPC). As such, other than the connectors, no additional circuit on the audio subsystem board is required for the batteries, fans, and/or LED rings.

In some embodiments, power is supplied from the charging port to support the audio subsystem in a charging state. FPC is used to connect the audio subsystem board and the mask board. The control buttons and indication LEDs are connected to the audio subsystem board via the FPC. FIG. 9B illustrates a diagram of example inner connection(s) on a printed circuit board for the smart mask.

Various FPC connectors may be used to connect the audio subsystem and the mask subsystem. Table-1 (below) illustrates a list of FPC connectors that may be included for connecting the audio subsystem and the mask subsystem.

TABLE 1

| Number | Signal name | Signal description |
|---|---|---|
| 1 | BOOT_1.8V | Mask board MCU reset by BT |
| 2 | ANC_key | Bluetooth ANC on/off key |
| 3 | GND | Common ground |
| 4 | BAT+ | Battery positive |
| 5 | BAT+ | Battery positive |
| 6 | FAN_FG | Fan speed control |
| 7 | FAN− | Fan supply, negative |
| 8 | FAN+ | Fan supply, positive |
| 9 | GND | Common ground |
| 10 | LED_Ring− | LED ring supply, negative |
| 11 | V_USB+ | Charging voltage from charging port, |
| | | to be used for BT subsystem |
| 12 | BAT_NTC | NTC sense from battery cell, to |
| | | connect to fan power board |
| 13 | GND | Common ground |
| 14 | USB_D+ | Positive pin of USB |
| 15 | USB_D− | Negative pin of USB |
| 16 | BAT+ | Battery positive |
| 17 | BAT+ | Battery positive |
| 18 | 1.8V | Power of buttons |
| 19 | RXD_1.8V | RX for UART |
| 20 | TXD_1.8V | TX for UART |

FIG. 9C illustrates an example FPC connector, having multiple pins. Table-2 below illustrates a list of pin sequences of the FPC connectors, corresponding to the pins of FIG. 9C.

TABLE 2

| Pin20 | Pin19 | Pin18 | Pin17 | Pin16 | Pin15 | Pin14 | Pin13 | Pin12 | Pin11 |
|---|---|---|---|---|---|---|---|---|---|
| TXD_1.8V | RXD_1.8V | 1.8V | BAT+ | BAT+ | USB_D− | USB_D+ | GND | BAT_NTC | V-USB+ |
| Pin1 | Pin2 | Pin3 | Pin4 | Pin5 | Pin6 | Pin7 | Pin8 | Pin9 | Pin10 |
| BOOT_1.8V | ANC_key | GND | BAT+ | BAT+ | FAN_FG | FAN− | FAN+ | GND | LED_Ring− |

Further, as illustrated in FIG. 9D, a female connector part is on the printed circuit board, and a male connector part is on the FPC connectors. FIG. 9E further illustrates a schematic diagram of the FPC connectors that connects the audio subsystem and the mask subsystem.

In some embodiments, the control buttons, including volume up/volume down, multi-function buttons, red/white LED indicator(s), are connected to the FPC connectors. FIG. 9F illustrates a schematic diagram of multiple control buttons connected to the FPC connectors. Table-3 below shows a list of example signals defined at the FPC connectors.

TABLE 3

| Number | Signal name | Signal description |
|---|---|---|
| 1 | 1.8V | Power for Volume Key |
| 2 | Vol-_key | Volume- key |
| 3 | BT_power_Key | BT Multi-function key |
| 4 | Vol+_key | Volume+ control |
| 5 | GND | GND |
| 6 | BAT+ | Power for the key |
| 7 | WHITE_LED | White LED |
| 8 | RED_LED | Red LED |
| 9 | GND | Common ground |
| 10 | GND | Common ground |

Table-4 below lists example definitions of a list of battery connector pins.

TABLE 4

| Number | Pin Name |
|---|---|
| 1 | GND |
| 2 | BAT_NTC |
| 3 | NC |
| 4 | BAT+ |

Table-5 below lists example definitions of a list of fan connector pins.

TABLE 5

| Number | Pin Name |
|---|---|
| 1 | FAN+ |
| 2 | FAN− |
| 3 | FAN_FG |

Table-6 below lists example definitions of a list of LED ring connector pins.

TABLE 6

| Number | Pin Name |
|---|---|
| 1 | LED_Ring− |
| 2 | BAT+ |

FIG. 9G illustrates an outline of a printed circuit board and its connectors. In some embodiments, there may be a multi-function control button on the printed circuit board configured as a power on/off control. In embodiments, there are multiple buttons on each of the left side and right side of the mask. On one side of the mask, there is a multi-function control button or ANC button. The audio subsystem can be turned on/off by long-pressing the multi-function control button for a predetermined time (e.g., 3 seconds). In some embodiments, the multi-function control button is configured to control the power of the audio subsystem only. In some embodiments, the multi-function control button is also configured to control ANC related functions, such as switching the audio subsystem between ANC mode, ambient mode, or off mode. In some embodiments, ANC and ambient modes may also be controlled by voice commands via Bluetooth to the audio subsystem board.

There are multiple buttons on another side of the mask for controlling the fan(s), ANC, and/or LED lights. The fan button may be configured to control various speeds of the fan. For example, a first press of the fan button causes the fan to run at a high speed, a second press of the fan button causes the fan to run at a medium speed, and a third press of the fan button causes the fan to run at a low speed, and a fourth press of the fan button causes the fan to power off. The ANC button is configured control the different ANC mode, ambient mode, and off mode. The LED button is configured to turn on or off the LED light or ring.

In some embodiments, two microphones are implemented. A first microphone is placed inside the mask towards the user for receiving the user's voice. In some embodiments, to avoid the direct breath from the user into the first microphone, the first microphone is positioned at one side of the mouth. A second microphone is configured to enable environment noise cancellation. The environment noise include noise from the fan or ambient noise (such as noise generated by wind, motor, or vehicle). In some embodiments, at least one of the microphones (especially the first microphone) is capable of working in a highly humid environment, due to the moisture generated by breath.

In some embodiments, there are three working modes associated with ANC functions, including an ANC mode, an ambient mode, and an off mode. Users can switch between the three modes by pressing the ANC button. In some embodiments, when the ANC mode is on, the noise reduction level is about 30 dB. In some embodiments, the ANC mode is the default mode when the earphone is powered up. The ambient mode may be used to provide additional awareness in an outdoor environment. When the ambient mode is on, external sound can be collected by a microphone and played from the speaker or earphone. For example, when a user is making a conversation with others, the user may choose to use the ambient mode without having to remove the earphone. The off mode is designed for users who may want to use the earphone as a regular Bluetooth earphone without ANC features. In the off mode, both the ANC and ambient functions are turned off.

In some embodiments, when a user presses the ANC button, a voice prompt is generated, notifying the user which mode is activated. Table-7 below illustrates the functions and voice prompts for different modes. In some embodiments, when the earphone is powered up, the ANC mode is on by default.

TABLE 7

| | ANC | Ambient | Voice prompt |
|---|---|---|---|
| Mode 1 | On | Off | “Noise cancellation on” |
| (ANC mode) | (default) | | |
| Mode 2 | Off | On | “Ambient on” |
| (Ambient mode) | | | |
| Mode 3 | Off | Off | “Off” |

Radio frequency (RF) performance of the audio subsystem includes working distance, connection stability, anti-interference, and cross body performance. In some embodiments, working distance in an open space is at least 15 meters, and the Bluetooth earphone is designed to be able to keep stable connections in a complex radio environment, having complex WiFi and Bluetooth coverage. Also, the audio subsystem is designed to have no connection issues when a user puts their phone in a pocket at any posture.

In some embodiments, the acoustic frequency range of the audio subsystem is in a range about 20 Hz to 20 KHz. In some embodiments, the speaker(s) or earphone(s) are dynamic speakers about 10.7 mm; the sensitivity of the speaker(s) or earphone(s) is about 113 dB SPL +/−3 dB at reference 1 mW at 1 Khz; total harmonic distortion (THD) is less than 1%; and impedance is about 32 Ohm. In some embodiments, the microphone(s) is omnidirectional. FIG. 9H illustrates a graph illustrating the sensitivity of an example speaker or earphone at different frequencies. FIG. 9I is a graph illustrating THD of an example speaker or earphone at different frequencies.

In some embodiments, a few different methods may be configured to cause the earphone to enter a pairing mode. For example, in some embodiments, when the earphone is powered up the first time, or after a factory reset, the earphone enters the pairing mode. In some embodiments, every time the earphone is powered up, the earphone first tries to reconnect to a previously paired device. If after a predetermined period (e.g., 15 seconds), the earphone still cannot reconnect to the previously paired device, the earphone enters the pairing mode automatically. In some embodiments, a user can press and hold the Bluetooth multi-function button for a predetermined time (e.g., 5 seconds) from the power off state to make the audio subsystem to enter the pairing mode. In embodiments, the pairing time is about 3-5 seconds. If a connection is lost in the middle of the connection due to link loss (e.g., weak signal), the Bluetooth audio subsystem may be configured to try to reconnect within a predetermined period (e.g., 10 minutes). If the earphone cannot be reconnected to the lost device within the predetermined period, the earphone may also enter the pairing mode. In some embodiments, when the earphone is in the pairing mode for a predetermined period (e.g., 90 seconds) without being able to pair with any device, the earphone is automatically powered off.

In some embodiments, users are allowed to restore the Bluetooth audio subsystem to a factory condition. For example, a user may press and hold both ANC button and volume up button for a predetermined period to cause the Bluetooth audio subsystem to reset to the factory condition. When the Bluetooth audio subsystem is reset to the factory condition, all the previous pairing histories are cleared. In some embodiments, the Bluetooth audio subsystem cannot be reset when a charger is plugged in, though the audio subsystem is able to work continuously when the charger is plugged in. In some embodiments, when the charger is plugged in, the audio subsystem maintains its current working state (i.e., without resetting, power on, or power off). After the charger is plugged in, a user is allowed to manually perform normal operations, including (but not limited to) power on or off, play music, or make phone calls.

In some embodiments, the control buttons for the Bluetooth audio subsystem may include multiple tact switches connected to the Bluetooth audio subsystem through FPC cable(s). In some embodiments, at least four buttons are formed on the mask for controlling the Bluetooth audio subsystem, including a volume up button, a volume down button, a multi-function button (for controlling play/pause/Bluetooth power), and an ANC button. Table-8 illustrates various statuses, operations, and actions of each of the control buttons.

TABLE 8

| Status | Operation | Action |
|---|---|---|
| Music | Single short press Multi-function | Play/pause |
| player | Double press Volume up | Next track |
| | Double press Volume down | Previous track |
| Ambient | Single long press ANC on/off | Loop between |
| Mode/ANC | | ANC, Ambient, |
| Mode/Off | | off |
| Incoming | Single short press Multi-function | Answer call |
| call | Double short press Multi-function | Reject call |
| During call | Single short press Multi-function | Hang up call |
| Volume | Single press Volume up | Volume up for 1 step |
| Control | Single press Volume down | Volume reduce for 1 |
| | | step |
| System | Press and hold Multi-function | System on/off |
| on/off | key for 3 seconds | |
| Force | Press and hold Multi-function | BT boot up enter |
| pairing | key for 5 seconds (from power | force pairing mode |
| | off state) | |
| Factory | Press and hold ANC on/off and | Factory reset, clear |
| reset | Volume up | all previous pairing |
| | | history |

In some embodiments, various voice prompts may be played accompanying the operations of the control buttons. For example, when the mask subsystem is powered on, a voice prompt of “mask on” may be played. When the audio subsystem enters a pairing mode, a voice prompt of “ready for pairing” may be played. When a device is paired, a voice prompt of “connected” may be played. When a connection is lost, a voice prompt of “disconnected” may be played. When the battery is low, a voice prompt of “battery low” may be played. When the ANC mode is on, a voice prompt of “noise cancellation on” may be played. When the ambient mode is on, a voice prompt of “ambient on” may be played. When the ANC mode and ambient mode are both off, a voice prompt of “noise cancellation off” may be played. In some embodiments, when the user adjusts the volume, a sound indication, such as a beep, is played. In some embodiments, when the maximum or minimum volume is reached, a certain sound (e.g., two beeps) may be played. In some embodiments, when the minimum volume is reached, and the user continues to press the volume down button, the headphone may be set to a silent mode.

In some embodiments, various LED indication logic may be implemented. For example, there may be a green LED on the wearer's left side underneath the buttons for fan, ANC button, and/or puck lights. The green LED may placed on the FPC cable that connects with the fan button, indicating the status of the fan. For example, when the fan is on, the green LED is on. There may also be different colored LEDs (e.g., a white LED and a red LED), indicating the status of the mask subsystem and Bluetooth audio subsystem. Table-9 below illustrates the operation corresponding to the LED lights indications.

TABLE 9

| Operation | BT light indication |
|---|---|
| BT power up | White LED blink once every 3 seconds |
| BT Pairing with phone | White and Red LED fast flicking every |
| | 0.3 seconds |
| BT Pairing done | White LED blink once every 10 seconds |
| Battery low | Red LED fast blink twice every 5 |
| | seconds. (when battery capacity below |
| | 20%, voice prompt every 5 mins) |
| BT power off | Red LED light for 1 second before |
| | power off |

Further, when the Bluetooth audio subsystem is connected to a smart device (e.g., a mobile phone), the Bluetooth audio subsystem may be controlled over-the-air (OTA) through a mobile app. For example, the Bluetooth audio subsystem may be updated via OTA firmware update. In some embodiments, the audio subsystem is configured to keep monitoring the battery level and sending the battery level to the connected smart device. When the battery level is lower than a predetermined threshold (e.g., 20%), a voice prompt of “battery low” may be played, and/or a red LED is caused to flash.

In some embodiments, the mask subsystem is configured to manage battery charging and protection, fan, LED ring driver, and the control thereof. FIG. 9J illustrates schematic diagrams of example circuits for battery management and protection. FIG. 9K illustrates schematic diagrams of example circuits for connecting the fan and LED to the FPC connector. FIG. 9L illustrates an example battery cell that may be embedded in the smart mask 100. The battery cell may have a capacity of about 735 mAh with nominal voltage of about 3.85 volts.

Because the power consumption of the audio subsystem is less than the mask subsystem, it is advantageous to place the main battery and charging circuit in the mask subsystem. The power of the audio subsystem is supplied from the mask subsystem. In some embodiments, the supply voltage is about 3.7 volts and/or in a range of about 3 volts and 4.6 volts, and the supply current is about 30 mA.

In some embodiments, there is no charging function on the audio subsystem. The battery charging is performed in the mask subsystem, though the earphone can continue working when the battery is being charged. When the battery is being charged, a charging cable (e.g., a USB charging cable) is inserted into the charging port. In some embodiments, the puck LED(s) blink one or more times to show that power has been established. The LED(s) may remain on until the power is fully charged (e.g., 100% charged); and when the battery is fully charged (i.e., 100% charged), the LED(s) are turned off.

FIGS. 9M and 9N illustrate both sides of an example printed circuit board (PCB) having various connectors. The PCB may include a 20-pin FPC connector, a battery connector, a fan connector, a LED ring connector, and an earbud connector. In embodiments, there is a 4 mm clearance in front of each connector in the connector plug direction.

FIGS. 10A-10C illustrate example mobile application user interfaces 1000A-1000C. FIG. 4A illustrates an example home page (or default view) 1000A of the mobile application. From the home page 1000A, a user can navigate to various control functions of the smart mask 100, 800. For example, the wearer can tap the heart rate icon 1010A to review his/her current heart rate. Alternatively, or in addition, the heart rate monitor is configured to record the wearer's heart rate over a period, and the wearer can tap the heart rate icon 1010A to review his/her heart rates over the period. Similarly, the wearer can tap the temperature icon 1020A to review his/her current body temperature or body temperatures over a period. Further, the wearer may tap the sound control icon 1030A, the air control icon 1040A, or the light control icon 1050A to enter a separate control interface for controlling the audio subsystem, the air circulation subsystem, and/or the light subsystem.

In some embodiments, an image 1060A of the smart mask may also be shown in the user interface 1000A, and the wearer may touch the different parts of the image 1060A of the smart mask to initiate the control of the corresponding component of the smart mask. For example, the wearer may touch the puck(s) shown on image 1060A to initiate the control function of the air circulation subsystem and/or the light subsystem. As another example, the wearer may also touch the earbud(s) to initiate the control function of the audio subsystem.

FIG. 10B illustrates an example air control interface 1000B that includes a toggle switch that allows a user to turn on or off the air circulation subsystem. The air control interface 1000B also includes a slider that allows the user to control the speed of the fan. In some embodiments, the user can move a bar to any point of the slider 1020B. In some embodiments, the user can move the bar to a few discrete places, such as at low, medium, high marks.

FIG. 10C illustrates another example user interface 1000C, which only has three control buttons, namely, a sound control button 1010C, an air control button 1020C, and a light control button 1010C. In some embodiments, each of the sound, air, light control buttons 1010C-1030C may be configured to simply turning on or off the sound, air, and/or light subsystem. In some embodiments, when each of the sound, air, light control buttons 1010C-1030C is touched, a separate control user interface is displayed, e.g., the user interface 1000B of FIG. 2. Similar to the user interface 1000A of FIG. 10A, the image 1060C of the smart mask in FIG. 10C may also be allowed to interact with the user.

In some embodiments, a wearer first pair the smart mask 100 with a mobile device via the mobile application. After the smart mask 100 is paired with the mobile device, the wearer may then stream music, make calls, and control in mask fans, LED lights, and/or ANC subsystem via the mobile application. In some embodiments, the wearer may also control sound including volume and equalization via the mobile application. In some embodiments, the mobile application may also generate real time notification for filter change and battery life. In some embodiments, the mobile application may also have a chatbot walkie-talkie mode that allows the user to use voice commands to control the smart mask 100 and/or interact with the mobile device.

Note, the user interfaces illustrated in FIGS. 10A-10C are merely schematic examples. Similar or different user interfaces may be implemented to achieve similar or different functions depending on the model and/or components of the smart mask.

Finally, because the principles described herein may be performed in the context of a computing system (for example, the one or more controller(s) o the smart masks may be a computing system, and the user terminal may be a computing system) some introductory discussion of a computing system will be described with respect to FIG. 11.

Computing systems are now increasingly taking a wide variety of forms. Computing systems may, for example, be handheld devices, appliances, laptop computers, desktop computers, mainframes, distributed computing systems, data centers, or even devices that have not conventionally been considered a computing system, such as wearables (e.g., glasses). In this description and in the claims, the term “computing system” is defined broadly as including any device or system (or a combination thereof) that includes at least one physical and tangible processor, and a physical and tangible memory capable of having thereon computer-executable instructions that may be executed by a processor. The memory may take any form and may depend on the nature and form of the computing system. A computing system may be distributed over a network environment and may include multiple constituent computing systems.

As illustrated in FIG. 11, in its most basic configuration, a computing system 1100 typically includes at least one hardware processing unit 1102 and memory 1104. The processing unit 1102 may include a general-purpose processor and may also include a field-programmable gate array (FPGA), an application-specific integrated circuit (ASIC), or any other specialized circuit. The memory 1104 may be physical system memory, which may be volatile, non-volatile, or some combination of the two. The term “memory” may also be used herein to refer to non-volatile mass storage, such as physical storage media. If the computing system is distributed, the processing, memory and/or storage capability may be distributed as well.

The computing system 500 also has thereon multiple structures often referred to as an “executable component”. For instance, memory 1104 of the computing system 1100 is illustrated as including executable component 1106. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed on the computing system, whether such an executable component exists in the heap of a computing system, or whether the executable component exists on computer-readable storage media.

In such a case, one of ordinary skill in the art will recognize that the structure of the executable component exists on a computer-readable medium such that, when interpreted by one or more processors of a computing system (e.g., by a processor thread), the computing system is caused to perform a function. Such a structure may be computer-readable directly by the processors (as is the case if the executable component were binary). Alternatively, the structure may be structured to be interpretable and/or compiled (whether in a single stage or in multiple stages) so as to generate such binary that is directly interpretable by the processors. Such an understanding of example structures of an executable component is well within the understanding of one of ordinary skill in the art of computing when using the term “executable component”.

The term “executable component” is also well understood by one of ordinary skill as including structures, such as hardcoded or hard-wired logic gates, that are implemented exclusively or near-exclusively in hardware, such as within a field-programmable gate array (FPGA), an application-specific integrated circuit (ASIC), or any other specialized circuit. Accordingly, the term “executable component” is a term for a structure that is well understood by those of ordinary skill in the art of computing, whether implemented in software, hardware, or a combination. In this description, the terms “component”, “agent”, “manager”, “service”, “engine”, “module”, “virtual machine” or the like may also be used. As used in this description and in the case, these terms (whether expressed with or without a modifying clause) are also intended to be synonymous with the term “executable component”, and thus also have a structure that is well understood by those of ordinary skill in the art of computing.

In the description that follows, embodiments are described with reference to acts that are performed by one or more computing systems. If such acts are implemented in software, one or more processors (of the associated computing system that performs the act) direct the operation of the computing system in response to having executed computer-executable instructions that constitute an executable component. For example, such computer-executable instructions may be embodied in one or more computer-readable media that form a computer program product. An example of such an operation involves the manipulation of data. If such acts are implemented exclusively or near-exclusively in hardware, such as within an FPGA or an ASIC, the computer-executable instructions may be hardcoded or hard-wired logic gates. The computer-executable instructions (and the manipulated data) may be stored in the memory 504 of the computing system 500. Computing system 500 may also contain communication channels 508 that allow the computing system 1100 to communicate with other computing systems over, for example, network 1110.

While not all computing systems require a user interface, in some embodiments, the computing system 1100 includes a user interface system 1112 for use in interfacing with a user. The user interface system 1112 may include output mechanisms 1112A as well as input mechanisms 1112B. The principles described herein are not limited to the precise output mechanisms 1112A or input mechanisms 1112B as such will depend on the nature of the device. However, output mechanisms 1112A might include, for instance, speakers, displays, tactile output, holograms and so forth. Examples of input mechanisms 1112B might include, for instance, microphones, touchscreens, holograms, cameras, keyboards, mouse or other pointer input, sensors of any type, and so forth.

Embodiments described herein may comprise or utilize a special purpose or general-purpose computing system including computer hardware, such as, for example, one or more processors and system memory, as discussed in greater detail below. Embodiments described herein also include physical and other computer-readable media for carrying or storing computer-executable instructions and/or data structures. Such computer-readable media can be any available media that can be accessed by a general-purpose or special purpose computing system. Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: storage media and transmission media.

Computer-readable storage media includes RAM, ROM, EEPROM, CD-ROM, or other optical disk storage, magnetic disk storage, or other magnetic storage devices, or any other physical and tangible storage medium which can be used to store desired program code means in the form of computer-executable instructions or data structures and which can be accessed by a general-purpose or special purpose computing system.

A “network” is defined as one or more data links that enable the transport of electronic data between computing systems and/or modules and/or other electronic devices. When information is transferred or provided over a network or another communications connection (either hardwired, wireless, or a combination of hardwired or wireless) to a computing system, the computing system properly views the connection as a transmission medium. Transmissions media can include a network and/or data links which can be used to carry desired program code means in the form of computer-executable instructions or data structures and which can be accessed by a general-purpose or special-purpose computing system. Combinations of the above should also be included within the scope of computer-readable media.

Further, upon reaching various computing system components, program code means in the form of computer-executable instructions or data structures can be transferred automatically from transmission media to storage media (or vice versa). For example, computer-executable instructions or data structures received over a network or data link can be buffered in RAM within a network interface module (e.g., a “NIC”), and then eventually transferred to computing system RAM and/or to less volatile storage media at a computing system. Thus, it should be understood that storage media can be included in computing system components that also (or even primarily) utilize transmission media.

Computer-executable instructions comprise, for example, instructions and data which, when executed at a processor, cause a general-purpose computing system, special purpose computing system, or special purpose processing device to perform a certain function or group of functions. Alternatively or in addition, the computer-executable instructions may configure the computing system to perform a certain function or group of functions. The computer executable instructions may be, for example, binaries or even instructions that undergo some translation (such as compilation) before direct execution by the processors, such as intermediate format instructions such as assembly language, or even source code.

Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, data centers, wearables (such as glasses) and the like. The invention may also be practiced in distributed system environments where local and remote computing system, which are linked (either by hardwired data links, wireless data links, or by a combination of hardwired and wireless data links) through a network, both perform tasks. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

The remaining figures may discuss various computing system which may correspond to the computing system 1100 previously described. The computing systems of the remaining figures include various components or functional blocks that may implement the various embodiments disclosed herein as will be explained. The various components or functional blocks may be implemented on a local computing system or may be implemented on a distributed computing system that includes elements resident in the cloud or that implement aspect of cloud computing. The various components or functional blocks may be implemented as software, hardware, or a combination of software and hardware. The computing systems of the remaining figures may include more or less than the components illustrated in the figures and some of the components may be combined as circumstances warrant. Although not necessarily illustrated, the various components of the computing systems may access and/or utilize a processor and memory, such as processor 502 and memory 504, as needed to perform their various functions.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A smart mask, comprising:
a face covering configured to cover a face area of a wearer;
an attachment member configured to attach the face covering onto the face area of the wearer;
one or more power source(s);
an air circulation subsystem powered by at least one of the one or more power source(s) and configured to filter outside air and draw the filtered outside air into the face area;
an exhaust subsystem placed below the air circulation subsystem and configured to purge exhaled air out of the face area; and
a controller configured to control the air circulation subsystem.

2. The smart mask of claim 1, wherein the air circulation subsystem comprises at least:
one or more filter receptacle(s), each of which is configured to receive a filter cartridge for filtering the outside air drawn into the face area;
one or more fan(s), each of which is powered by at least one of the one or more power source(s) and configured to draw outside air through a corresponding filter cartridge into the face area; and
one or more puck(s), each of which is configured to removably cover the corresponding filter cartridge or fan.

3. The smart mask of claim 2, wherein the one or more fan(s) include a left fan placed on a left side of the face area and a right fan placed on a right side of the face area;
the one or more filter receptacle(s) includes (1) a left filter receptacle coupled to the left fan and configured to receive a left filter cartridge, and (2) a right filter receptacle coupled to the right fan and configured to receive a right filter cartridge; and
the one or more puck(s) includes a left puck configured to cover the left fan and the left filter cartridge, and a right puck configured to cover the right fan and the right filter cartridge.

4. The smart mask of claim 2, wherein each of the one or more filter receptacle(s) comprises a magnetic portion, configured to hold a metal frame of the filter cartridge in place magnetically.

5. The smart mask of claim 2, wherein each of the one or more filter receptacle(s) has a circular-shaped recess configured to receive a circular-shaped filter cartridge.

6. The smart of claim 2, wherein each of the one or more fan(s) is configured to operate at different speeds, and the controller is configured to control a speed of the one or more fan(s).

7. The smart mask of claim 2, further comprising:
one or more ultraviolet light sources powered by at least one of the one or more power source(s) and configured to disinfect an area around the filter cartridge, wherein the controller is further configured control the one or more ultraviolet light source(s).

8. The smart mask of claim 7, wherein each of the one or more ultraviolet light source(s) is coupled to a corresponding one of the one or more puck(s) or a corresponding one of the one or more filter receptacle(s).

9. The smart mask of claim 8, wherein each of the one or more ultraviolet light source(s) is configured to emit an ultraviolet light at different intensities, and the controller is further configured to control an intensity of the one or more ultraviolet light source(s).

10. The smart mask of claim 1, further comprising:
an audio subsystem powered by at least one of the one or more power source(s) and configured to receive a sound signal wirelessly from a user terminal; and
the controller is further configured to control the audio subsystem.

11. The smart mask of claim 10, wherein the audio subsystem includes at least (1) a pair of earbuds or (2) a pair of speakers.

12. The smart mask of claim 11, wherein each of the pair of earbuds includes a magnetic portion or a metal portion,
the attachment member also includes a pair of magnetic portions or metal portions corresponding to the pair of earbuds, such that when the pair of earbuds are not in use, each of the pair of earbuds is configured to be secured to the corresponding magnetic portion or metal portion of the attachment member magnetically.

13. The smart mask of claim 10, wherein the audio subsystem further includes a microphone configured to receive a voice input, and the controller is further configured to process the voice input to identify one or more voice command(s) for controlling the air circulation subsystem or the audio subsystem.

14. The smart mask of claim 1, further comprising:
a network interface configured to connect to a user terminal, wherein when the user terminal is connected to the network interface, the network interface is configured to transmit status data to the user terminal, and to grant the user terminal control of the air circulation subsystem.

15. The smart mask of claim 1, further comprising:
a temperature sensor configured to detect a body temperature of the wearer.

16. The smart mask of claim 1, further comprising:
a heart rate monitor configured to detect a heart rate of the wearer.

17. The smart mask of claim 1, wherein the one or more power source(s) include one or more rechargeable batteries, and the smart mask further comprises a charging port configured to charge the one or more rechargeable batteries.

18. A filter cartridge configured to fit a face mask having a fan and a receptacle for receiving a filter, comprising:
a plurality of layers of fabric; and
a metal frame configured to snap onto a magnetic portion of the receptacle of the face mask.

19. The filter cartridge of claim 18, further comprising:
an aperture in a center of the plurality of layers of fabric to allow a nose of the fan of the face mask to protrude through.

20. A smart mask, comprising:
a face covering configured to cover a face area of a wearer;
an attachment member configured to attach the face covering onto the face of the wearer;
one or more power source(s);
an air circulation subsystem powered by at least one of the one or more power source(s) and configured to filter outside air and draw filtered outside air into the face area;
one or more ultraviolet light source(s) powered by at least one of the one or more power source(s) and configured to disinfect an area of the face covering around the wearer's mouth;
an exhaust subsystem configured to purge exhaled air out of the face area; and
a controller configured to control the air circulation subsystem and the one or more ultraviolet light source(s).
